(19) United States
(12) Patent Application Publication
(10) Pub. No.: US 2026/0139986 A1
(43) Pub. Date: May 21, 2026

(54) LIQUID LEVEL SENSOR

(71) Applicant: Ecolab USA Inc., Saint Paul, MN (US)

(72) Inventors: Eugene Tokhtuev, Woodbury, MN (US); Brock Stephen Mueggenborg, Saint Paul, MN (US)

(21) Appl. No.: 19/391,447

(22) Filed: Nov. 17, 2025

Related U.S. Application Data
(60) Provisional application No. 63/722,468, filed on Nov. 19, 2024.

(51) Int. Cl. G01F 23/24; G01F 1/69; G01K 1/12; G01K 7/24; G08B 21/18

(52) U.S. Cl. CPC G01F 23/248; G01F 1/69; G01K 1/12; G01K 7/24; G08B 21/182

(57) ABSTRACT

A liquid level sensor can include a circuit board and a thermistor bridge supported by the circuit board comprising a first pair of thermistors and a second pair of thermistors. A heat sink can be coupled to the circuit board proximate the first pair of thermistors and not the second pair of thermistors. A power supply can be configured to provide pulses of electrical current through the thermistor bridge. A controller in communication with the thermistor bridge can be configured to receive a measurement signal from the thermistor bridge output a low product indication when the measurement signal satisfies a predetermined condition.

### RELATED APPLICATION

This application claims priority to U.S. Provisional Application No. 63/722,468, filed Nov. 19, 2024, the entire contents of which are incorporated herein by reference.

### BACKGROUND

Fluid dispensing systems typically deliver quantities of fluid to one or more components within the system. In certain fields, fluid dispensing systems may deliver small quantities of fluid. For example, in the medical field, a fluid dispensing system may be used to deliver small quantities of fluid into a patient's vascular system. However, in certain other fields, fluid dispensing systems may deliver larger quantities of fluid. For example, in a large-scale hotel or other laundry or restaurant facility, a fluid dispensing system may need to deliver large quantities of detergent, rinse agent, bleach or other cleaning agents on a continual basis.

In fluid delivery systems where large quantities of fluid are delivered, the fluid can be supplied automatically. In such systems, the supply source (such as a bottle) and fluid delivery line (such as a supply tube) are frequently integrated with the device to which the fluid is delivered, such as a warewasher or a laundry machine. This makes it more difficult for the operator to check on the remaining amount of the fluid remaining in the supply source and often results in the system running out of fluid during, for example, a cleaning cycle.

### SUMMARY

Various aspects of this disclosure relate to fluid flow systems. A fluid flow system can include a liquid level sensing system comprising a liquid level sensor and a controller in communication with the liquid level sensor. In some embodiments, the liquid level sensor can include a circuit board, a thermistor bridge supported by the circuit board, the thermistor bridge comprising a first pair of thermistors and a second pair of thermistors, and a heat sink coupled to the circuit board proximate the first pair of thermistors and not the second pair of thermistors. The system can include a power supply configured to provide pulses of electrical current through the thermistor bridge. The controller can be in communication with the thermistor bridge and configured to receive a measurement signal from the thermistor bridge and output a low product indication when the measurement signal satisfies a predetermined condition.

The predetermined condition can indicate that a level of liquid product in a reservoir (e.g., in the fluid flow system) falls below a predetermined level. The level can be set, for example, so that the controller outputs a low product indication when there is sufficient product remaining in the reservoir for a predetermined number of system operating steps (e.g., sufficient product remaining for one complete wash phase in a warewashing fluid flow system).

A liquid product in a product reservoir can affect the thermal behavior of the thermistors proximate the liquid product. Pulsing electrical current through the thermistor bridge can cause the thermistors to increase in temperature, and a measurement signal from the thermistor bridge can provide insight into whether the first and second pairs of thermistors are behaving thermally similarly or differently.

The first pair of thermistors and second pair of thermistors can be positioned such that the second pair of thermistors are above the first pair of thermistors. When the level of liquid product is above both pairs of thermistors, it thermally affects both pairs of thermistors similarly. As liquid product from a reservoir is depleted, the level of liquid will drop below the second pair of thermistors while still being proximate the first pair of thermistors so that the liquid product thermally affects the first pair of thermistors more than the second. A difference in thermal behavior between the first and second pair of thermistors can indicate that the liquid level has dropped below the second pair of thermistors but not the first pair of thermistors.

As the liquid product level drops below the level of the first pair of thermistors, the heat sink proximate the first pair of thermistors can cause the first pair of thermistors to continue to behave thermally differently than the second pair of thermistors. This can prevent false indications that the level of liquid product is above the first and second pair of thermistors due to the two pairs of thermistors behaving thermally similarly.

Liquid level sensing information can be used in combination with out of product sensor information to provide added insight into fluid flow systems. An out of product sensor indicating no liquid is flowing while a liquid level sensor indicates a high level of liquid in a reservoir could indicate an issue with the pump, control system, or wiring. If the out of product sensor indicates no liquid flowing for a period of time but then detects flowing liquid, there could be a priming issue with the delivery of the fluid and product dosing can be adjusted to be counted from the time that flow was detected rather than when a pump was activated. If the liquid level sensor indicates a high level of liquid and the out of product sensor senses product flowing, the system is operating as intended. If the liquid level sensor changes to detect a low amount of product, this can indicate that product is running out and should be addressed. The system can be configured to output a warning that the level of product is low.

### BRIEF DESCRIPTION OF THE DRAWINGS

FIG. 1A is a diagram illustrating an example fluid flow system with a liquid level sensing system that detects a relative level of a product to be dispensed.

FIG. 1B is a diagram illustrating another example system that dispenses multiple products.

FIGS. 2A-2C show an example liquid level sensor configurations.

FIG. 3A shows an example liquid level sensor that can be inserted into a product reservoir

FIG. 3B shows a layout of an example liquid level sensor with a product reservoir.

FIG. 4 shows an example schematic diagram of aspects of a liquid level sensing system.

FIG. 5 is an example voltage vs. time plot showing a voltage output from a switch showing an example measurement cycle.

FIG. 6 shows an example plot of a measurement signal of a liquid level sensor at different product levels within a reservoir.

FIG. 7 shows an example system including a liquid level sensor and configured to provide an indication of a level of liquid in a product reservoir.

FIG. 8 is a diagram illustrating an example fluid flow system with a liquid level sensing system that detects a relative level of a product to be dispensed and an out of product (OOP) sensor configured to detect a presence of a flowing liquid product.

FIG. 9A shows a side view of an example configuration of an out of product sensor.

FIG. 9B shows a top view of the out of product sensor of FIG. 9A.

FIG. 10A shows an example cross-sectional view of an embodiment of an OOP sensor.

FIG. 10B shows a perspective exploded view of the OOP sensor of FIG. 10A.

FIG. 10C shows another perspective exploded view of the OOP sensor of FIG. 10A.

FIG. 11 shows an example plot of a measurement signal over time that can be used to determine a fluid flow status through an out of product sensor.

### DETAILED DESCRIPTION

The following detailed description is exemplary in nature and is not intended to limit the scope, applicability, or configuration of the disclosure in any way. Rather, the following description provides some practical illustrations for implementing examples of the present disclosure. Examples of constructions, materials, dimensions, and manufacturing processes are provided for selected elements, and all other elements employ that which is known to those of ordinary skill in the field of the disclosure. Those skilled in the art will recognize that many of the noted examples have a variety of suitable alternatives.

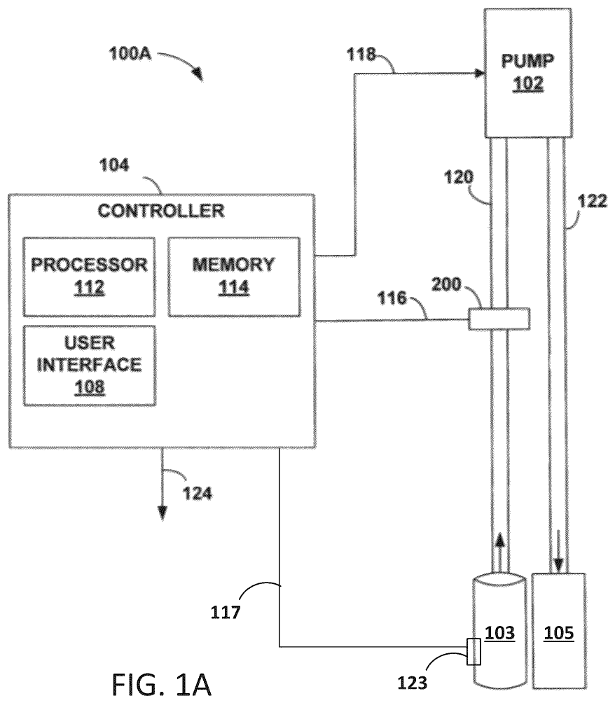

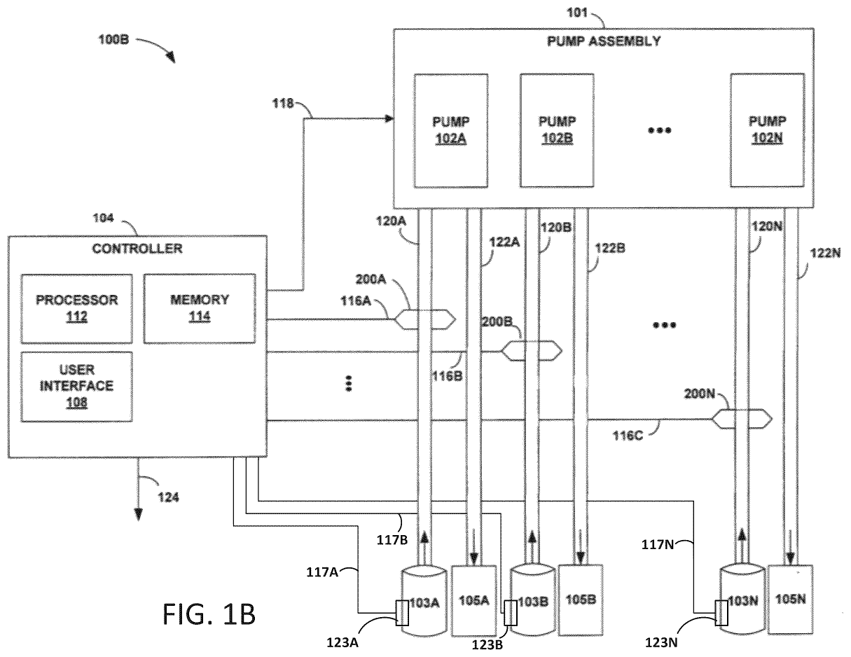

FIG. 1A is a diagram illustrating an example fluid flow system with an out-of-product sensing system that detects presence and/or absence of a product to be dispensed. The system 100A includes controller 104, pump 102 and product reservoir 103. Pump 102 draws the product (e.g., a liquid product) from reservoir 103 and delivers the product to dispensing site 105. Pump 102 draws product from product reservoir 103 through an input fluid delivery line 120 and supplies fluid to dispensing site 105 via an output fluid delivery line 122. Product reservoir 103 may contain any one of a multitude of different types of products having varying degrees of transparency and/or turbidity.

In some embodiments, an out of product (“OOP”) sensor 200 is configured to detect a presence or absence of a fluid flowing, for example, in input fluid delivery line 120 and/or output fluid delivery line 122. In the illustrated examples, OOP sensor 200 is positioned in line with the input fluid delivery line 120, and can be configured to determine, for example, a presence or absence of a fluid (e.g., a liquid) in the line.

The system of FIG. 1A further includes a liquid level sensor 123 associated with reservoir 103. In some examples, the liquid level sensor 123 is configured to provide information about an amount of liquid in reservoir 103. In various embodiments, liquid level sensor 123 can be positioned inside or outside of the reservoir 103. In some examples, liquid level sensor 123 is configured to detect a level of liquid in reservoir 103 relative to one or more threshold levels. For example, in some embodiments, liquid level sensor 123 is configured to output a signal indicative of whether the level of liquid in reservoir 103 is above or below a predetermined threshold level. In various examples, the liquid level sensor 123 can output information regarding the level of liquid in reservoir 103 compared to one or more thresholds (e.g., above or below a first predetermined threshold, above or below a second predetermined threshold, etc.). This can include, for example, outputting a first signal when a liquid level is above a first predetermined threshold, outputting a second signal when a liquid level is between the first predetermined threshold and a second predetermined threshold lower than the first, and a third signal when the liquid level is below the second predetermined threshold. In some examples, predetermined thresholds can be predetermined relative to the liquid level sensor 123 itself. In some such examples, the level of liquid in the reservoir corresponding to one or more predetermined thresholds is a function of the placement of the liquid level sensor 123 relative to the reservoir 103. In other examples, liquid level sensor 123 can be integral with the reservoir 103, such that a predetermined threshold level relative to the liquid level sensor 123 is also predetermined relative to the reservoir 103.

Controller 104 can communicate with pump 102 via connection 118. In some examples, pump 102 draws the product from reservoir 103 or stops pumping under the control of controller 104. In some examples, controller 104 may communicate with dispensing site 105 via another connection (not shown).

In some examples, controller 104 includes processor 112, user interface 108, and memory 114. In some examples, systems can include multiple controllers 104. Signals generated by OOP sensor 200 can be communicated to controller 104 via connection 116. Connection 116 may transmit a digital or analog signal. Connection 116 may include, for example, a standard I2C connection. However, any appropriate connection/communication channel known in the art may be used. Additionally or alternatively, signals generated by liquid level sensor 123 can be communicated to controller 104 via connection 117. Connection 117 may transmit a digital or analog signal. Connection 117 may include, for example, a standard I2C connection. However, any appropriate connection/communication channel known in the art may be used. Controller 104 can further include at least one external connection 124 such as an internet, telephone, wireless or other connection for achieving external communication.

In some examples, memory 114 stores software for running controller 104 and also stores data that is generated or used by processor 112. In some examples, processor 112 runs software stored in memory 114 to manage operation of controller 104. User interface 108 may be as simple as a few light emitting diodes (LEDs) and/or user actuatable buttons or may include a display, a keyboard or keypad, mouse or other appropriate mechanisms for communicating with a user.

Dispensing site 105 may be an end use location of the product or may be some other intermediate location. For example, when a fluid flow system 100A is used in a commercial laundry or kitchen application, dispensing site 105 may be a washing machine or warewashing machine, in which case the product(s) may be dispensed into an on-unit dispense mechanism or directly into the wash environment. In that example, the product(s) dispensed may include laundry or dish detergent, fabric softener, bleach, sanitizer, rinse agent, etc. As another example, when fluid dispensing system is used in a hotel, business, industrial or other application in which service employees perform cleaning duties, dispensing site 105 may be a bucket, pail or other vessel into which the product(s) are dispensed. Dispensing site 105 may also be a hose or other tubing from which the fluid(s) is directed to a desired location. It shall be understood that an out-of-product sensing system can be used in many different applications in which fluid is dispensed and that the disclosure is not limited in this respect. Examples of applications in which an out-of-product sensing system can be used include laundry applications, dishwashing applications, commercial cleaning operations, food preparation and packaging applications, industrial processes, healthcare applications, vehicle care applications, and others known in the art.

Input fluid delivery line 120 and output fluid delivery line 122 may be implemented using any type of flexible or inflexible tubing, depending upon the application. This tubing may be transparent, translucent, braided or other type of tubing. The tubing may be made of polyethylene, ethylene-vinyl acetate, polytetrafluoroethylene, or any other suitable material. For simplicity and not by limitation, input fluid delivery line 120 and output fluid delivery line can be referred to as “input tubing 120” and “output tubing 122,” respectively. Input tubing 120, output tubing 122 and pump 102 may be referred to herein as a “dispensing channel.” Pump 102 may be any form of pumping mechanism that supplies fluid from product reservoir 103 to dispensing site 105. For example, pump 102 may comprise a peristaltic pump or other form of continuous pump, a positive-displacement pump or other type of pump appropriate for the particular application.

In the example system shown in FIG. 1A, OOP sensor 200 is positioned to detect presence and/or absence of product within input tubing 120. In operation, when fluid dispensing system attempts a dispensing cycle from a product reservoir 103 that has product remaining, input tubing 120 will likewise contain product. In some examples, OOP sensor 200 continuously sends signals to controller 104, and controller 104 interprets those signals to determine product presence or absence within input tubing 120. Over time, as operation continues and more and more product is dispensed, product reservoir 103 becomes substantially empty. Because product is no longer available to dispense, input tubing 120 will likewise become substantially empty. When controller 104 determines that an out-of-product event has occurred based on the signals from OOP sensor 200, controller 104 may generate an out-of-product alert.

In some embodiments, an “out-of-product event” (e.g., an event in which controller 104 detects an absence of fluid within input tubing 120) is determined with respect to one or more predefined out-of-product thresholds. When controller 104 detects an out-of-product event, controller 104 may generate one or more alerts, including a visual and/or audible out-of-product alert (such as text or graphics with without accompanying sound, etc.) displayed on user interface 108. Additionally or alternatively, controller 104 may initiate and send an out-of-product message service call (such as via pager, e-mail, text message, etc.) to a technical service provider via external connection 124.

When an alert is activated to indicate an out-of-product event, a user (such as an employee or service technician) may manually refill product reservoir 103. In this embodiment, the user may temporarily halt or shutdown operation of the fluid flow system before refilling product reservoir 103. In one example, the user may do this by entering commands into controller 104 to stop operation of pump 102 and/or dispensing site 105. In another example, the user may do this by entering control commands via user interface 108 of controller 104 to silence audible and/or visual alerts for a period of time. In another example, the user may do this by entering control commands via user interface 108 of controller 104 to stop operation of pump 102 and/or dispensing site 105. In another example, the user may manually shut off pump 102 and/or dispensing site 105. After the user has refilled product reservoir 103, the user may manually re-start pump 102 and/or dispensing site 105, may enter control commands into controller 104 to restart pump 102 and/or dispensing site 105, or may enter control commands via user interface 108 to cause controller 104 to send control signals (e.g., via connection 118) to re-start pump 102 and/or dispensing site 105. Controller 104 may further re-set, or clear, alerts at the appropriate time (for example, after being manually cleared by a user, after product reservoir 103 has been refilled or system is restarted).

In response to an out-of-product event, controller 104 can automatically stop pump 102 and/or dispensing site 105 when an out-of-product event is detected and/or automatically stop dispensing site 105. In one example, controller 104 may send control signals to pump 102 and/or dispensing site 105 to temporarily stop operation of the corresponding components without user intervention. Controller 104 may then re-start pump 102 and/or dispensing site 105 after receiving input from the user that product reservoir 103 has been re-filled. In another example, controller 104 can temporarily stop pump 102 and/or dispensing site 105 without user intervention. System controller can then send signals to re-start pump 102 and/or dispensing site 105 after receiving input from the user that product reservoir 103 has been re-filled.

OOP sensor 200 or controller 104 may also generate a visual indicator that indicates presence of fluid within input tubing 120. For example, a light of one color, such as green, may be used to indicate that a fluid is flowing through the OOP sensor 200, indicating that product reservoir 103 has product remaining, while a light of another color, such as red or blinking, may be used to indicate that fluid is not flowing through OOP sensor 200, indicating that product reservoir 103 is empty and needs to be refilled.

In addition to or alternatively to detecting out of product events via the OOP sensor 200, in some examples, controller 104 can be configured to determine when an amount of product in reservoir 103 falls below one or more predetermined levels based on liquid level sensor 123. In some cases, one or more such predetermined levels occurs before the OOP sensor 200 is likely to detect an out-of-product event as product in reservoir 103 is depleted over time. For instance, in some embodiments, liquid level sensor 123 is positioned at a level relative to the bottom of the reservoir 103 such that, even after the product level in the reservoir 103 drops below a predetermined level, sufficient product remains in the reservoir 103 for pump 102 to draw product therefrom. In some embodiments, the liquid level sensor 123 is configured such that the controller can determine an amount of product in the reservoir 103 relative to one or more levels, such as whether the amount or product is above a first level, below a first level but above a second level, lower than the first, or below the second level.

In the example system shown in FIG. 1A, liquid level sensor 123 is positioned to detect when a level of a product in reservoir 103 is above or below one or more predetermined levels. In operation, when fluid dispensing system performs a dispensing cycle from a product reservoir 103, the product level in the reservoir 103 will drop. The liquid level sensor 123 can be configured to output a first signal when the product level is above a predetermined level, and output a second signal when the product level is below the predetermined level. In some examples, the liquid level sensor is configured to output the first signal when the product level is above a first predetermined level, output the second signal when the product level is below the first predetermined level but above a second predetermined level lower than the first, and output a third signal when the product level is below the second predetermined level.

In some examples, liquid level sensor 123 continuously sends signals to controller 104, and controller 104 interprets those signals to determine whether the amount of product in reservoir 123 is above or below one or more predetermined levels. Over time, as operation continues and more and more product is dispensed, the product in product reservoir 103 eventually falls below one or more predetermined levels, changing the output of the liquid level sensor 103, for example, from the first signal to the second signal when the product level falls below a first predetermined level and from the second signal to the third signal when the product falls below the second predetermined level. The controller 104 can be configured to interpret this change in output from the liquid level sensor to determine that the level of product in the reservoir 103 has fallen below the first or second predetermined level. When controller 104 determines that the level of product in the reservoir 103 has fallen below a predetermined level, controller 104 may generate a low product alert. In some examples, the controller 104 can output different levels of low product alert, for example, depending on which predetermined level(s) the product level is below.

In some embodiments, a “low product event” (e.g., an event in which controller 104 detects the product within reservoir 103 falling below a predetermined level with respect to the liquid level sensor 123) is determined with respect to one or more predefined low product thresholds. In some cases, multiple low product events are possible as a product level within the reservoir 103 continues to fall. For example, a first low product event can correspond to a product level falling below a first predetermined level and a second low product event can correspond to a product level falling below a second predetermined level lower than the first. When controller 104 detects a low product event, controller 104 may generate one or more alerts, including a visual and/or audible low product alert (such as text or graphics with without accompanying sound, etc.) displayed on user interface 108. Additionally or alternatively, controller 104 may initiate and send a low product message service call (such as via pager, e-mail, text message, etc.) to a technical service provider via external connection 124. In some examples, different responses can be performed for different low product events.

In some examples, when an alert is activated to indicate a low product event, a user (such as an employee or service technician) may manually refill product reservoir 103. In this embodiment, the user may temporarily halt or shutdown operation of the fluid flow system before refilling product reservoir 103. In one example, the user may do this by entering commands into controller 104 to stop operation of pump 102 and/or dispensing site 105. In another example, the user may do this by entering control commands via user interface 108 of controller 104 to silence audible and/or visual alerts for a period of time. In another example, the user may do this by entering control commands via user interface 108 of controller 104 to stop operation of pump 102 and/or dispensing site 105. In another example, the user may manually shut off pump 102 and/or dispensing site 105. After the user has refilled product reservoir 103, the user may manually re-start pump 102 and/or dispensing site 105, may enter control commands into controller 104 to restart pump 102 and/or dispensing site 105, or may enter control commands via user interface 108 to cause controller 104 to send control signals (e.g., via connection 118) to re-start pump 102 and/or dispensing site 105. Controller 104 may further re-set, or clear, alerts at the appropriate time (for example, after being manually cleared by a user, after product reservoir 103 has been refilled or system is restarted).

In some examples, in response to a low level event, controller 104 can automatically stop pump 102 and/or dispensing site 105 when a low level event is detected and/or automatically stop dispensing site 105. In one example, controller 104 may send control signals to pump 102 and/or dispensing site 105 to temporarily stop operation of the corresponding components without user intervention. Controller 104 may then re-start pump 102 and/or dispensing site 105 after receiving input from the user that product reservoir 103 has been re-filled. In another example, controller 104 can temporarily stop pump 102 and/or dispensing site 105 without user intervention. System controller can then send signals to re-start pump 102 and/or dispensing site 105 after receiving input from the user that product reservoir 103 has been re-filled.

Liquid level sensor 123 or controller 104 may also generate a visual indicator that indicates a level of product remaining in reservoir 103, at least relative to one or more predetermined levels. For example, a light of one color, such as green, may be used to indicate that the amount of product in the reservoir 103 is above a first predetermined level, while a light of another color, such as yellow, may be used to indicate that the amount of product in the reservoir is below the first predetermined level but above a second predetermined level, and another color or indication, such as red or blinking, may be used to indicate that the amount of product in the reservoir 103 is below the second predetermined level. In some examples, the indication that the product above the first predetermined level indicates that there is sufficient product in reservoir 103 for operation, the indication that the product level is below the first predetermined level but above the second predetermined level indicates that the reservoir will need to be refilled soon, for example, after one or more process cycle using the product from the reservoir, and the indication that the product level is below the second predetermined level indicates that product reservoir 103 is nearly empty and should not be used until refilled.

In some embodiments, liquid level sensor 123 can be integrated into a housing of reservoir 103. In other examples, liquid level sensor 123 can be attached to a housing of the reservoir 103, such as inside or outside of the reservoir 103. In still other examples, the liquid level sensor can be a separate sensor not attached to any housing of the reservoir 103. For instance, in some examples, the liquid level sensor can be supported by a probe inserted into the reservoir 103 or included on a portion of tubing 120 inserted into the reservoir 103.

FIG. 1B is a diagram illustrating another example system that dispenses multiple products. To that end, system 100B includes multiple product channels (A-N), each having associated product reservoirs 103A-103N, pumps 102A-102N, controller 104 and dispensing sites 105A-105N. Pumps 102A-102N are included in pump assembly 101. Pumps 102A-102N draw in fluid from a respective product reservoir 103A-103N through an input tubing 120A-120N, and supply fluid to one of dispensing sites 105A-105N through output tubing 122A-122N. Each product reservoir 103A-103N may contain any of a multitude of different types of products. OOP sensors 200A-200N detect presence and/or absence of the product dispensed in the respective each dispensing channel. Each product reservoir 103A-103N further includes a corresponding liquid level sensor, 123A-123N, respectively, which can output a signal to controller 104 providing information regarding an amount of product in each corresponding product reservoir 103A-103N, such as whether an amount of product in each product reservoir 103A-103N is above or below a predetermined amount. In some examples, the predetermined amount for each product reservoir 103A-103N is the same predetermined amount, while in other examples, the predetermined amount for each product reservoir 103A-103N need not be the same.

Although the system 100B shown in FIG. 1B shows each dispensing channel as having its own dedicated product reservoir 103, input tubing 120, output tubing 122, pump 102, destination site 105, OOP sensor 200, and liquid level sensor, it shall be understood that there need not be a one to one correspondence for each dispensing channel. For example, sensors 200A-200N may be implemented in a single unit through which the input tubing for each dispensing channel is routed. Alternatively, various combinations of one channel per sensor or two or more channels per sensors may also be used and the disclosure is not limited in this respect. In some cases, a single reservoir can provide product to multiple dispensing sites, and a single liquid level sensor can be used to provide information representative of the amount of product in the single reservoir.

The example pump assembly 101 of FIG. 1B includes multiple pumps 102A-102N, one for each dispensed product. It shall be understood, however, that there need not be a one to one correspondence between pumps 102A-102N and the dispensing channels. For example, some dispensed products may share one or more pumps, which are switched from one dispensed product to another under control of controller 104. The pump or pumps 102A-102N provide fluid to the appropriate dispensing site 105A-105N from one of product reservoirs 103A-103N.

It shall also be understood that any of sensors 200A-200N may also be positioned to detect presence and/or absence of product within output tubing 122A-122N rather than input tubing 120A-120N as shown in FIG. 1B, and that, in some cases, the location of sensors 200A-200N may be more a matter of convenience than of system performance.

In some examples, controller 104 can be coupled to pump assembly 101 via connection 118. Through connection 118, controller 104 is able to communicate with pump assembly 101 to effectively control operation of each individual pump 102 (e.g., to temporarily stop or start operation, as described previously in reference to FIG. 1A). Depending upon the application, controller 104 may also communicate with one or more dispensing sites 105A-105N.

Each OOP sensor 200A-200N detects presence and/or absence of fluid within the corresponding input tubing 120A-120N. Controller 104 is coupled to each sensor 200A-200N via a corresponding connection 116A-116N. Controller 104 monitors the signals received from each OOP sensor 200A-200N, and may respond as described above to any detected out-of-product events. For example, controller 104 may generate a visual or audible alert or display a message on user interface 108 if system controller detects one or more out-of-product events. The visual or audible alert and/or message displayed on user interface 108 and/or message sent via pager, e-mail or text message, etc. would indicate which of product reservoirs 103A-103N is empty, thus informing a user which product reservoir needs to be filled. In some examples, controller 104 may also automatically temporarily stop and then re-start the pump 102A-102N corresponding to the empty product reservoir 103A-103N and/or may initiate an automatic refill cycle of the empty product reservoir as described above. In other examples, pumps 102A-102N and/or dispensing sites 105A-105N may be stopped and re-started automatically or manually, with or without communication from controller as described with respect to FIG. 1A above.

Although in FIG. 1B each sensor assembly is shown with a dedicated connection to controller 104, it shall be understood that sensors 200A-200N may be connected to communicate with controller 104 in any of several different ways. For example, sensors 200A-200N may be connected to controller 104 in a daisy-chain fashion. In this example, controller 104 is coupled directly to a first OOP sensor 200A via connection 116A and each subsequent OOP sensor 200B-200N is coupled the next sensor assembly, etc. A communication protocol to identify and communicate separately with each OOP sensor 200A-200N may also be used. It shall be understood, however, that this disclosure is not limited with respect to the particular architecture by which sensors 200A-200N are connected with and communicate with controller 104 and that the system may be set up in many different ways known to those of skill in the art.

Each liquid level sensor 123A-123N can be configured to detect whether an amount of product in each corresponding product reservoir 103A-103N is above or below a corresponding predetermined amount. Controller 104 is coupled to each sensor 123A-123N via a corresponding connection 117A-117N. Controller 104 monitors the signals received from each liquid level sensor 123A-123N, and may respond as described above to any detected low product events. For example, controller 104 may generate a visual or audible alert or display a message on user interface 108 if system controller detects one or more low product events. The visual or audible alert and/or message displayed on user interface 108 and/or message sent via pager, e-mail or text message, etc. would indicate in which of product reservoirs 103A-103N an amount of product has fallen below a predetermined level, thus informing a user which product reservoir needs to be filled. In some examples, controller 104 may also automatically temporarily stop and then re-start the pump 102A-102N corresponding to the product reservoir 103A-103N for which the amount of product has fallen below the predetermined amount and/or may initiate an automatic refill cycle of the low product reservoir as described above. In other examples, pumps 102A-102N and/or dispensing sites 105A-105N may be stopped and re-started automatically or manually, with or without communication from controller as described with respect to FIG. 1A above.

Although in FIG. 1B each sensor assembly is shown with a dedicated connection to controller 104, it shall be understood that sensors 123A-123N may be connected to communicate with controller 104 in any of several different ways. For example, sensors 123A-123N may be connected to controller 104 in a daisy-chain fashion. In this example, controller 104 is coupled directly to a first liquid level sensor 123A via connection 117A and each subsequent liquid level sensor 123B-123N is coupled the next sensor assembly, etc. A communication protocol to identify and communicate separately with each liquid level sensor 123A-123N may also be used. It shall be understood, however, that this disclosure is not limited with respect to the particular architecture by which liquid level sensors 123A-123N are connected with and communicate with controller 104 and that the system may be set up in many different ways known to those of skill in the art.

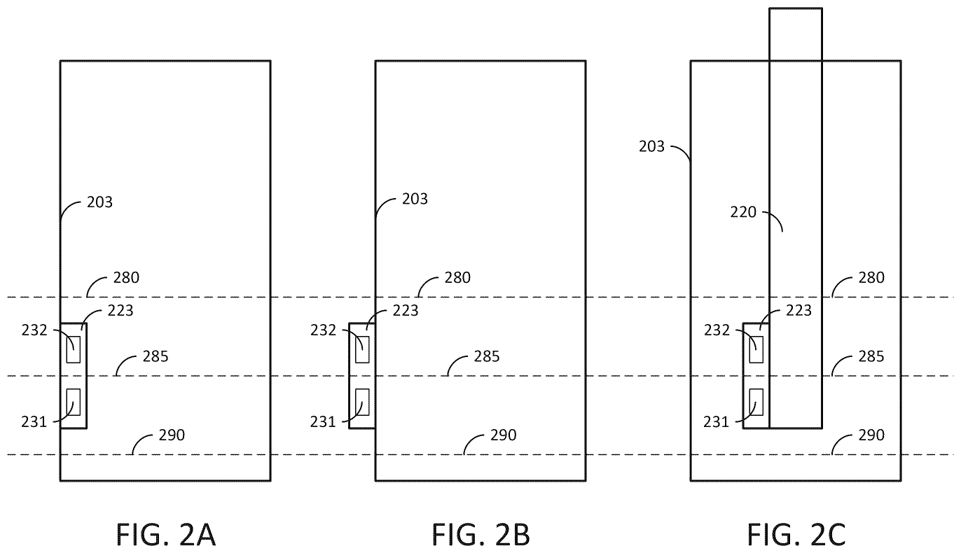

FIG. 2A shows an example liquid level sensor 223 positioned on an interior wall of a reservoir 203. The liquid level sensor 223 has a first sensing area 231 and a second sensing area 232. In some examples, the liquid level sensor can be used to detect information about a level of liquid product in the reservoir 203 relative to the sensing areas. For example, when the level of product in the reservoir 203 is at 280, the liquid level sensor 223 can be configured to output a first signal indicating that the level of product is above a first predetermined level. In some examples, the first predetermined level is approximately the level associated with the second sensing area 232.

In some examples, when the level of product in reservoir 203 is at 285, between the first sensing area 231 and the second sensing area 232, the liquid level sensor 223 can be configured to output a second signal different from the first signal and that indicates that the level of product is lower than the first predetermined level and above a second predetermined level, lower than the first. In some examples, the second predetermined level is approximately the level associated with the first sensing area 231.

In some examples, when the level of product in reservoir 203 is at 290, below the first sensing area 231, the liquid level sensor 223 can be configured to output a third signal different from the first signal the second signal and that indicates that the level of product is lower than the second predetermined level.

In some examples, a liquid level sensor can use effects of a liquid product on thermal behavior of the sensor to determine information about the amount of liquid product in a reservoir. For example, when the level of product in the reservoir 203 is at level 280, the liquid can thermally affect the first sensing area 231 and the second sensing area 232. When the level of product in the reservoir 203 is at level 285, the liquid can thermally affect the first sensing area 231, but has less or no thermal effect on the second sensing area 232. And when the level of product in the reservoir 203 is at level 290, the liquid has little or no thermal effect on either the first sensing area 231 or the second sensing area 232. This variability in thermal effects on the first and/or second sensing areas can be used to provide information about the level of product in the reservoir 203 relative to the sensing areas.

As shown in FIG. 2A, liquid level sensor 223 can be positioned on an interior surface of the reservoir 203. The liquid level sensor can include one or more sensors (e.g., thermistors). The sensors can be shielded from the product in the reservoir 203 such that the sensors are not directly exposed to the product, but can be thermally affected by the product.

FIG. 2B shows a configuration where liquid level sensor 223 is positioned outside of the reservoir 203. The liquid level sensor 223 includes first sensing area 231 and second sensing area 232, and can operate in a similar way as shown in FIG. 2A. However, in some examples, the liquid product in the reservoir 203 thermally affects the sensing areas through the walls of the reservoir 203.

FIG. 2C shows a configuration where liquid level sensor 223 is positioned on tubing 220 within the reservoir 203. In some examples, as described elsewhere herein, tubing 220 can be used to draw liquid product from the reservoir 203. The tubing 220 can be used to support the liquid level sensor 223, which can include a first sensing area 231 and a second sensing area 232, and can operate in s similar way as shown in FIG. 2A. In some cases, the liquid level sensor 223 can be similarly supported by a probe inserted into the reservoir 203 rather than by tubing 220.

As shown in the examples of FIGS. 2A-2C, liquid level sensor 223 can be positioned relative to the reservoir 203 in a variety of ways while generally operating using similar principles. In the examples of FIGS. 2A-2C, a first sensing area 231 and second sensing area 232 are positioned at approximately a same level relative to the reservoir 203 and can operate in similar ways.

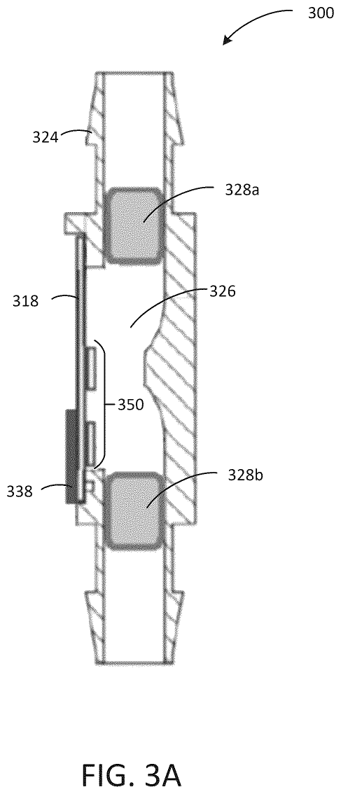

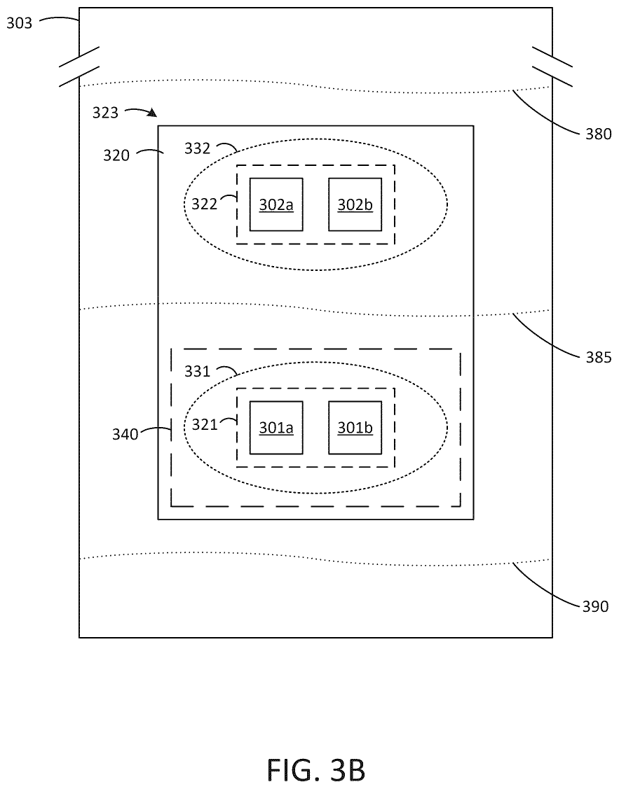

FIG. 3A shows an example liquid level sensor that can be inserted into a product reservoir. In the example embodiment, liquid level sensor 300 includes a housing 324 having an interior 326. The interior 326 can be closed off on opposite ends of the housing by plugs 328a, 328b. An opening in a sidewall of the housing can be closed off by a circuit board 318 (e.g., a printed circuit board). The circuit board 318 can support thermistors 350 and a heat sink 338 proximate one or more thermistors or thermistor pairs, such as described herein. Plugs 328a, 328b closing off ends of the housing 324 and the circuit board 318 covering an opening in a sidewall of the housing 324 can prevent liquid product from accessing the interior 326 of the sensor 300 and electrical components supported by a side of the circuit board 318 facing the interior 326, such as thermistors 350.

FIG. 3B shows a layout of an example liquid level sensor with a product reservoir. Liquid level sensor 323 of FIG. 3B is generally positioned within or otherwise adjacent reservoir 303. For instance, such as described with respect to FIGS. 2A-2C, in some examples, liquid level sensor can be within the reservoir, such as attached to or embedded in a wall of the reservoir or attached to a probe or tubing extending into the reservoir 303. In other examples, the liquid level sensor 323 can be attached to or embedded in an external surface of the reservoir 303.

The sensor 323 includes a circuit board 320 configured to support one or more sensors. In some examples, the circuit board 320 supports a thermistor bridge comprising a plurality of thermistors. In some embodiments, a liquid product within reservoir 303 thermally interacts with portions of the thermistor bridge supported by the circuit board 320 such that the fluid affects the temperature of one or more thermistors of the thermistor bridge. In some examples, liquid level sensor 323 is configured such that the circuit board 320 is positioned between thermistors and the liquid product within the reservoir 303. The circuit board 320 can be sufficiently thin and/or thermally conductive so that the liquid product within the reservoir 303 affects the temperature of thermistors through the circuit board 320. In some examples, circuit board is between approximately 0.025 mm thick and 0.25 mm thick. In some examples, the circuit board comprises a glass epoxy laminate FR-4 or polyimide. Additionally or alternatively, in some cases, thermistors can be positioned on the liquid product facing side of the circuit board 320, and a protective coating (e.g., a polyacrylic or other material) can coat the thermistors to protect the thermistors from the liquid product while allowing the liquid product to affect the thermal behavior of the thermistors.

In the example of FIG. 3B, the circuit board 320 supports first 301a, second 302b, third 302a, and fourth 301b thermistors. In some embodiments, the first thermistor 301a and the fourth thermistor 301b form a first pair of thermistors 321, and the second thermistor 302b and the third thermistor 302a form a second pair of thermistors. In some embodiments, the first pair of thermistors 321 are positioned in a first sensing area 331 and the second pair of thermistors 322 are positioned in a second sensing area 332. In the illustrated example, as discussed elsewhere herein, a heat sink 340 is positioned proximate the first sensing area 331. The heat sink 340 can include, for example, a copper, stainless steel, for other metal foil attached to the circuit board opposite first pair of thermistors 321. Additionally or alternatively, in some examples, heat sink 340 can include a metal or other layer attached to the circuit board near and on the same side as the first pair of thermistors 321. In various examples, the heat sink foil may have a thickness of between 0.025 mm and 0.25 mm and lateral dimensions of between 2 mm×3 mm and 10 mm×15 mm.

In an example embodiment, first thermistor 301a and fourth thermistor 301b of the first pair of thermistors 321 are separated by approximately 2 mm, and third thermistor 302a and second thermistor 302b of the second pair of thermistors 322 are separated by approximately 2 mm. In some examples, the first pair of thermistors 321 are separated from the second pair of thermistors 322 by a distance between approximately 4 mm and 10 mm.

Fluid can be present in reservoir 303 at various levels. At example liquid level 380, the liquid level is above both the first pair of thermistors 321 and the second pair of thermistors 322. Each pair of thermistors is affected by the liquid in the reservoir 303 approximately equally. However, at liquid level 385, the first pair of thermistors 321 are affected by the liquid more than the second pair of thermistors 322 are, which are generally above the liquid level 385. At liquid level 390, all of the thermistors 301a, 301b, 302a, 302b are above the level and are affected by the fluid in the reservoir approximately equally. However, in the presence of heat sink 340, the first pair of thermistors 321 may dissipate heat more quickly due to heat sink 340.

During example operating, thermistors 301a, 301b, 302a, 302b can be heated and allowed to cool such as described elsewhere herein. Liquid product in the reservoir, if thermally affecting one or more thermistors, can prevent such thermistor(s) from rising to as high of a temperature compared to thermistor(s) not thermally affected by the liquid product and/or can cause such thermistor(s) to cool more rapidly than those not thermally affected by the liquid product. Observing the thermal behavior of the thermistors can provide information about which thermistors are thermally affected by the liquid product in the reservoir 303, and such information can be used to determine information about the level of product in the reservoir 303.

In some examples, a heat sink 340 is positioned proximate the first sensing area 331. In some embodiments, the heat sink 340 affects the thermal behavior of the first pair of thermistors 321 in similar way as liquid product. When the heat sink is present the adding of liquid has low influence on heat dissipation properties of the first pair of thermistors 321 because the heat already efficiently dissipated by the heat sink. In some such examples, when a liquid product level is above the second sensing area 332 (e.g., at level 380), the first pair of thermistors 321 and the second pair of thermistors 322 thermally behave similarly due to the presence of fluid proximate the second pair of thermistors 322 and fluid and heat sink 340 proximate the first pair of thermistors 321.

When the liquid product level is between the first and second sensing areas (e.g., at level 385), there is an air and not a liquid near the second pair of thermistors 322, so the thermal behavior of the second pair of thermistors changes compared to when the liquid product is at, for example, level 380. However, compared to level 380, at level 385, the thermal behavior of the first pair of thermistors is not significantly changed because for each of level 380 and level 385 there is no difference in heat dissipation of the surroundings of the first pair of thermistors 321. Observable differences in the thermal behavior of the first pair of thermistors 321 and second pairs of thermistors 322 can indicate that the level of liquid product is between the first and second sensing areas (e.g., at level 385).

When the liquid product level is below the first sensing area (e.g., at level 390), the liquid product does not affect the thermal behavior of either pair of thermistors. However, the heat sink 340 causes the first pair of thermistors 321 to behave thermally differently from the second pair of thermistors 322. This helps prevent false indications that enough product remains in the reservoir to cover both the first and second sensing areas. For instance, in some cases, in the absence of heat sink 340, the first pair of thermistors 321 and second pair of thermistors 322 would behave thermally similarly when a liquid product is at either level 380 or 390, which could cause a false indication that there is sufficient product in the reservoir 303 to cover both the first and second sensing areas despite the level being at or even below level 390. The heat sink 340 can cause the first pair of thermistors 321 to continue to behave thermally differently than the second pair of thermistors 322 as the liquid level drops from, for example, 385 to and below level 390.

The liquid level sensor 323 can be configured to output a signal representative of the relative thermal behavior of the first and second pairs of thermistors. In some such examples, a first output can indicate that liquid product is affecting the thermal behavior of both pairs of thermistors and a second output can indicate that liquid product is affecting the thermal behavior of the first pair of thermistors 321 but not the second pair of thermistors 322. In some examples, a third output can indicate that the liquid product is not affecting the thermal behavior of either pair of thermistors, but the heat sink 340 is affecting the first pair of thermistors 321 such that the first pair of thermistors 321 thermally behaves differently than the second pair of thermistors 322. In some such embodiments, the third output is different from the first output and the second output. In other such embodiments, the third output can be approximately equal to the second output.

In some embodiments, the thermistors 301a, 301b, 302a, 302b can be arranged in a thermistor bridge configuration having two branches arranged in parallel. Power can be applied to the thermistor bridge such that electrical current travels through both branches. The electrical current can heat the thermistors. Since the resistance of a thermistor changes with its temperature, different thermal behavior (e.g., different temperatures at a given time) of various thermistors can affect the resistance of the thermistors, which can be detectable by measuring one or more voltages.

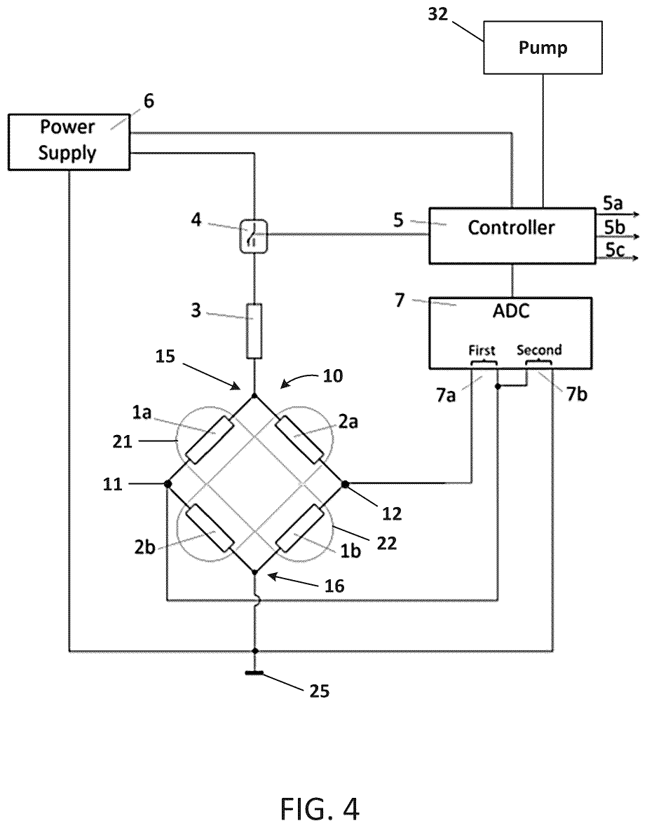

FIG. 4 shows an example schematic diagram of aspects of a liquid level sensing system. In the illustrated example, a thermistor bridge 10 comprises a plurality of thermistors, including a first thermistor 1a, a second thermistor 2b, a third thermistor 2a, and a fourth thermistor 1b. In the illustrated example, the thermistor bridge 10 comprises a first branch comprising the first thermistor 1a in series with the second thermistor 2b, with a first point 11 between the first thermistor 1a and the second thermistor 2b. The thermistor bridge 10 of FIG. 4 further comprises a second branch comprising the third thermistor 2a in series with the fourth thermistor 1b, with a second point 12 between the third thermistor 2a and the fourth thermistor 1b. In some examples, as described elsewhere herein, the first thermistor 1a and the fourth thermistor 1b for a first pair of thermistors 21 and the second thermistor 2b and the third thermistor 2a form a second pair of thermistors 22.

As shown, the first branch and the second branch are arranged in parallel between a powered side 15 of the thermistor bridge 10 and a reference side 16 of the thermistor bridge 10. In the illustrated example, the first thermistor 1a and the third thermistor 2a are coupled to the powered side 15 of the thermistor bridge 10 and the second thermistor 2b and the fourth thermistor 1b are coupled to the reference side 16 of the thermistor bridge 10.

The example system of FIG. 4 includes a power supply 6 coupled to the powered side 15 of the thermistor bridge 10 via a switch 4 and a current limiting resistor 3. In some examples, power supply 6 comprises a DC power supply configured to output a DC voltage. In some examples, the power supply 6 is configured to output a constant voltage, such as 5 VDC. In other examples, the power supply 6 can have a controllable output. The reference side 16 of the thermistor bridge 10 is coupled to a reference potential 25, such as a system ground. In various examples, switch 4 can include any type of switch capable of selectively interrupting current flow, such as a mechanical switch or a transistor. During operation, if switch 4 is in an on state, current can flow from the power supply 6 through switch 4 and current limiting resistor 3 to the powered side 15 of the thermistor bridge 10, and through the branches of the thermistor bridge 10 to the reference side 16.

The system of FIG. 4 includes an analog to digital converter (ADC) 7 having a first differential input 7a and a second differential input 7b. In the example of FIG. 4, the first input 7a of ADC 7 comprises inputs coupled to first point 11 and second point 12 of the thermistor bridge 10. Thus, in some examples, first input 7a of ADC 7 is configured to receive a signal representative of the voltage difference between the first point 11 and the second point 12. Additionally, in the example of FIG. 4, the second input 7b of ADC 7 comprises inputs coupled to second point 12 of the thermistor bridge 10 and a reference potential 25. Thus, in some examples, second input 7b of ADC 7 is configured to receive a signal representative of the voltage difference between the second point 12 and a reference voltage.

The system of FIG. 4 includes a controller 5 in communication with the ADC 7, the switch 4, and the power supply 6. In some examples, the controller 5 is configured to receive a measurement signal value representative of a voltage between the first point 11 and the second point 12, for example, from the ADC 7.

Additionally or alternatively, in some embodiments, controller 5 is configured to control operation of switch 4, for example, to control when current is permitted to or prevented from flowing between power supply 6 to thermistor bridge 10. Additionally or alternatively, controller 5 is configured to control operation of power supply 6, for example, enabling/disabling output of power from power supply 6 and/or adjusting an output of power supply 6.

In the illustrated example, controller 5 includes three outputs: a digital output 5a, and analog output 5b, and a logic output 5c. In various examples, controller 5 can include one or more such outputs, but need not necessarily provide all three. In some examples, the controller 5 is configured to provide an output based on information representative of a voltage between the first point 11 and the second point 12 of the thermistor bridge 10, such as, for example, received from ADC 7.

In some examples, controller 5 is in communication with a pump 32, which can be configured to cause fluid to flow through a fluid flow system, such as, for example, pump 102 in FIG. 1A configured to cause fluid to flow from a reservoir 103 to a dispensing site 105. Reservoir can include a corresponding liquid level sensor. In some examples, pump 32 is configured to cause fluid to flow through an out of product sensor. In some examples, controller 5 is configured to control operation of the pump 32, for example, turning the pump on and off and/or controlling a speed of the pump. In other examples, the controller is configured to receive information from the pump 32, such as an operating state (e.g., on/off) of the pump 32.

In various examples, controller 5 can include a general purpose microprocessor, digital signal processor (DSP), application specific integrated circuit (ASIC), field programmable gate array (FPGA), programmable logic devices (PLDs), or other equivalent logic devices, or combinations of one or more such components. In some examples, functions described herein attributed to a controller can be performed by one or more controllers. In some examples, systems can include multiple controllers distributed throughout a system acting in concert.

In some examples, controller 5 includes or is otherwise in communication with a memory, which can include instructions (e.g., in a non-transitory computer readable medium) for causing the controller to perform one or more functions. In some examples, memory comprises random access memory (RAM) such as synchronous dynamic random access memory (SDRAM), read-only memory (ROM), non-volatile random access memory (NVRAM), electrically erasable programmable read-only memory (EEPROM), embedded dynamic random access memory (eDRAM), static random access memory (SRAM), flash memory, magnetic or optical data storage media, or combinations of one or more such components.

FIG. 4 illustrates an example connection of thermistors 1a, 1b, 2a, 2b to the power supply 6. Typically, when thermistors are utilized to measure temperature, current passing through them is regulated to restrict power dissipation within the thermistor. Thermistors alter their resistance in response to temperature changes. In such cases, the resistance reflects the ambient temperature.

In some examples of operating a liquid level sensor according to the present disclosure, a high current pulse is briefly applied to the thermistors, causing a momentary change in their temperature. In some embodiments, this temperature variation can exceed 20° C. and, in some instances, reach up to or above 100° C. In some examples, the thermistors increase in temperature between 20° C. and 125° C.

In an example embodiment, thermistors 1a, 1b, 2a, 2b employed in a liquid level sensor are NTC thermistors, which lower their resistances when ambient temperature rises or when thermistor temperature rises during self-heating. When a constant voltage V is applied to the NTC thermistor, the dissipated power P initially starts from P₀=V²/Rₜ₀, where Rₜ₀ represents the resistance at the beginning. Nonetheless, the heightened temperature lowers the thermistor's resistance, causing the current to escalate over time unless the applied voltage is diminished or turned off. The inclusion of a constant resistor (e.g., current limiting resistor 3) in series with the thermistors safeguards them from potential damage.

As depicted in the electrical circuit diagram of FIG. 4, the thermistors 1a, 1b, 2a, 2b are arranged in a bridge 10, and a current limiting resistor 3 is connected in series. The selection of nominal resistance values for the thermistors, the limiting resistor, and the applied voltage is strategic, allowing rapid self-heating without endangering the thermistors. In some configurations, self-heating (e.g., application of a voltage from power supply 6) elevates the temperature of all thermistors similarly. This uniformity in temperature results from the short duration of preheating and the more gradual nature of heat transfer. Consequently, the thermistors exhibit similar temperatures during the self-heating process. Once the voltage is deactivated, the thermistors' temperatures begin to decline as heat dissipates into the surroundings. The rates of heat transfer can vary when different materials are in proximity to the thermistors, enabling the differentiation between fluids and air. As described elsewhere herein, short pulses of voltage applied to the thermistor bridge can be used to allow reading signals from one or more thermistors without heating it. As temperatures of one or more thermistors of the thermistor bridge 10 change, resulting changes in thermistor resistances can result in different signals received, for example, at ADC 7.

In an example embodiment, each of thermistors 1a, 1b, 2a, and 2b have the same resistance vs. temperature relationship. In such an example, if all thermistors are the same temperature and same resistance, then the voltage drop across thermistor 1a and thermistor 2a will be the same, and the voltage difference between points 11 and 12 will be zero. If thermistors 1a and 1b are the same temperature and thermistors 2a and 2b are the same temperature, and thermistors 1a and 1b have lower resistance than thermistors 2a and 2b, for example, due to temperature effects of a fluid in a reservoir affecting the thermal behavior of some but not all thermistors, then thermistor 1a will drop less voltage than thermistor 2a and the voltage difference between points 11 and 12 will be non-zero. Thus, in some examples, deviations in temperature between a first sensing area and a second sensing area can result in changes in the voltage difference between points 11 and 12, such as measured at input 7a of ADC 7 in FIG. 4. In some examples, a signal at input 7b (representative of a voltage drop across thermistor 2b) represents a general temperature level of the thermistors in the second sensing area.

With reference to FIG. 4, controller 5 is configured to place the switch 4 into an on state to cause current to flow from the power supply 6 to the thermistor bridge 10 to heat one or more thermistors of the thermistor bridge 10. Current from the power supply 6 flows through both the first branch and the second branch of the thermistor bridge to the reference potential. In some examples, the current through thermistors causes the temperature of the thermistors to rise. Fluid present in a reservoir and proximate one or more thermistors can affect the temperatures of the thermistors differently, for example, affecting thermistors in a first sensing area more than thermistors in a second sensing area.

In some examples, controller is configured to place the switch into an on state to cause current to flow from the power supply to the thermistor bridge for a heating time duration. In some examples, heating time duration is between 1 ms and 1000 ms. In some examples, the controller 5 is configured to place the switch 4 into an off state to stop current flowing to the thermistor bridge 10 and maintain the switch in the off state for a delay time duration. In some examples, delay time duration is between 1 ms and 1000 ms. After the delay time duration and during a reading time duration, the controller 5 is configured to provide a plurality of measurement pulses to the thermistor bridge 10, for example, by transitioning the switch 4 between an off and on state. In some embodiments, reading time duration is between 10 ms and 2000 ms. In some examples, each measurement pulse has a measurement pulse time duration (e.g., a time the switch is in the on state) of between 0.1 ms and 5 ms and being provided at a measuring pulse frequency of between 10 Hz and 100 Hz. For instance, in some examples, the time between the leading edge of consecutive pulses is between 10 ms and 100 ms.

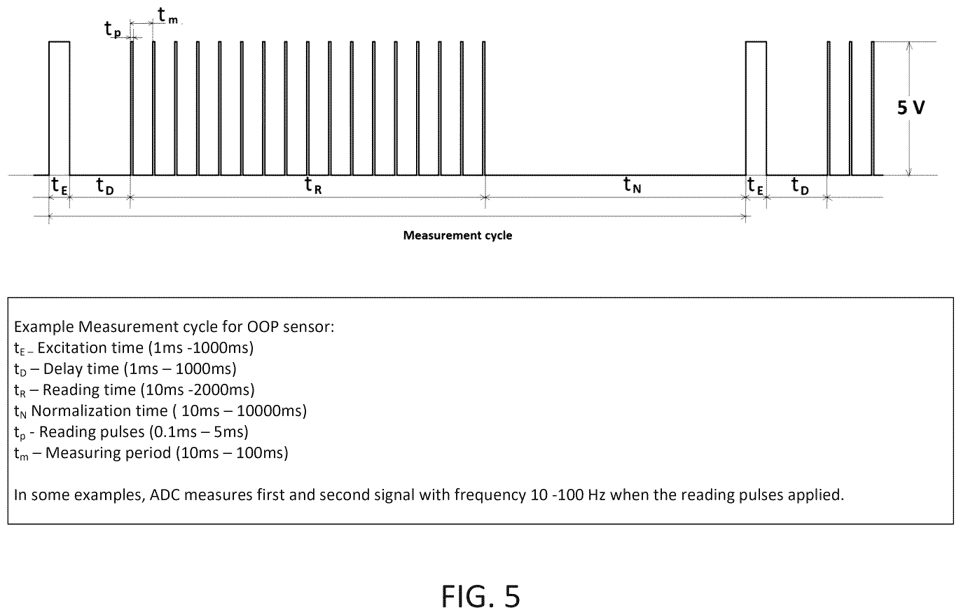

FIG. 5 is an example voltage vs. time plot showing a voltage output from switch 4. For instance, in some examples, the power supply 6 is configured to output 5 VDC and switch controls whether 5 VDC is output from switch. In the illustrated example, controller 5 controls switch 4 to provide power to the thermistor bridge 10 during an excitation time tE, to stop providing power for a delay time tD, to provide a series of excitations pulses during reading time tR, each measurement pulse having a reading pulse time tp, the leading edge of each pulse being separated by a measuring period tm. After a series of measurement pulses during reading time, the controller 5 can cause switch 4 to stop providing power to the thermistor bridge 10 for a normalization time tN. During the normalization time, thermistors can return to an equilibrium temperature. In some examples, the excitation time, delay time, reading time, and normalization time combine to form a measurement cycle. In some examples, measurement cycle can be repeated over time.

The controller 5 can be configured to, during each of the plurality of measurement pulses, receive a measurement signal value. In some examples, the measurement signal value is representative of a voltage between points 11 and 12 of the thermistor bridge 10. In some examples, the measurement signal value is an output from ADC 7 in communication with points 11 and 12. In some examples, the measurement signal is a voltage. In some examples, the measurement signal corresponds to a temperature difference between thermistors in the first sensing area and the second sensing area.

In some embodiments, the measurement pulses are sufficiently short so as to not significantly change the temperature of the thermistor(s), but is sufficiently long to determine a measurement signal value during the measurement pulse. In some examples, current limiting resistor 3 suppresses current flowing through the thermistors during the measurement pulses to prevent heating of the thermistors. Additionally or alternatively, in some examples, current limiting resistor 3 suppresses current flowing through the thermistors during the heating time duration to prevent damage to the thermistors. In some examples, power supply 6 provides a 5 VDC output. In some examples, current limiting resistor is between approximately 10 ohms and 100 ohms. In some examples, the resistance of thermistors range between approximately 30 ohms and 100 ohms across a range of operating temperatures.

In some embodiments, controller 5 is further configured to receive a second signal representative of a voltage drop across one thermistor (e.g., representative of a signal received as second input 7b of ADC 7 related to a voltage drop across thermistor 1b). In some examples, the second signal provides an indication of the temperature of the thermistors in the first sensing area. In some examples, the temperature of such thermistors can be used to make a temperature correction of the measurement signal value. Thus, in some examples, a measurement pulse is used to receive a measurement signal value based on a voltage drop between points 11 and 12 in FIG. 4, and the controller can be configured to receive a second signal representative of the voltage drop across thermistor 2b. Controller 5 can use the second signal representative of a temperature of the second sensing area to determine a corrected measurement signal value. For example, corrections (e.g., polynomial corrections) can be used to compensate for hot or cold fluid in the reservoir.

With reference to FIG. 3B, in some examples, the first thermistor 301a, fourth thermistor 301b, third thermistor 302a, and second thermistor 302b can be electrically configured in the thermistor bridge 10 shown in FIG. 4, in the places of first thermistor 1a, fourth thermistor 1b, third thermistor 2a, and second thermistor 2b, respectively. In such a configuration, first 301a and fourth 301b thermistors form a first branch of the thermistor bridge and third 302a and second 302b thermistors form a second branch of the thermistor bridge in parallel with the first. The thermistors 301a, 301b, 302a, 302b arranged in such a bridge can be operated such as described above.

In an example implementation, if the level of the liquid product in reservoir 303 is at level 380, the liquid product affects the thermal behavior of both the first pair of thermistors 321 and the second pair of thermistors 322 approximately equally. As a result, the temperatures of the first pair of thermistors 321 and the second pair of thermistors 322 will be approximately equal, and the voltage difference between points 11 and 12 will be low when power is provided to the thermistor bridge by power supply 6. The low voltage measurement signal can correspond to a first output of the liquid level sensor, corresponding to a situation when the liquid level is above a first predetermined level (e.g., above both the first 331 and second 332 sensing areas). In some cases, heat sink 340 has little or no appreciable effect on the thermal behavior of the first pair of thermistors 321 when the liquid product is also affecting the thermal behavior.

Continuing with the example implementation, if the level of the liquid product in reservoir 303 is at level 385, the liquid product affects the thermal behavior of the first pair of thermistors 321 but not the second pair of thermistors 322. For example, in some cases, after applying power to the thermistor bridge and heating the thermistors, the liquid product can cause the first pair of thermistors 321 to cool more quickly than the second pair of thermistors 322. Additionally or alternatively, the first pair of thermistors might not reach as high of a temperature as the second pair of thermistors.

In some examples, the comparatively lower temperature of thermistors 301a, 301b cause such thermistors to have a different resistance compared to thermistors 302a, 302b. A measurement pulse to the thermistor bridge in such a situation causes a resulting voltage between points 11 and 12 in the bridge, which can be measurable at controller 5. For instance, in an example embodiments, thermistors are negative temperature coefficient (NTC) thermistors. High temperature thermistors 302a, 302b will have lower resistance compared to thermistors 301a, 301b. If thermistors 301a, 301b, 302a, 302b are mapped into electrical locations of thermistors 1a, 1b, 2a, 2b in FIG. 4, respectively, thermistor 302a will, having lower resistance, drop less voltage than thermistor 301a, having a higher resistance, resulting in measurement signal having a positive voltage between points 12 and 11 at input 7a.

The positive voltage measurement signal can correspond to a second output representative of the liquid level in reservoir 303 being below the first predetermined level, but above a second predetermined level (below the second sensing area 332 but above the first sensing area 331). As product is depleted from the reservoir and the product level falls below the first predetermined level, the output from the sensor transitions from the first output to the second output. In some such examples, the measurement signal at input 7a transitions from a low value to a positive voltage.

Continuing with the example implementation, if the level of the liquid product in reservoir 303 is at level 390, the liquid product affects the thermal behavior neither the second pair of thermistors 322 nor the first pair of thermistors 321. However, heat sink 340 can affect the thermal behavior of the first pair of thermistors 321.

If, for example, after applying power to the thermistor bridge and heating the thermistors, the heat sink 340 can cause the first pair of thermistors 321 to cool more quickly than the second pair of thermistors 322 and/or not reach as high of a temperature as the second pair of thermistors 322. In some examples, the comparatively lower temperature of thermistors 302a, 302b cause such thermistors to have a different resistance compared to thermistors 301a, 301b. Similar as discussed above with respect to fluid level 385, a measurement pulse to the thermistor bridge in such a situation causes a measurement signal with a resulting voltage between points 11 and 12 in the bridge at input 7a. The resulting measurement signal can correspond to a third output representative of the liquid level in reservoir 303 being below the first predetermined level and the second predetermined level. In some examples, the heat sink 340 has less effect on the thermal behavior of the first pair of thermistors 321 than the liquid product (e.g., when liquid product is at level 385), such that the third output is distinguishable from the second output (e.g., a lower positive voltage between points 12 and 11 at input 7a compared to when the liquid is at level 385). As product is depleted from the reservoir and the product level falls below the second predetermined level, the measurement signal from the sensor transitions from the second output to the third output.

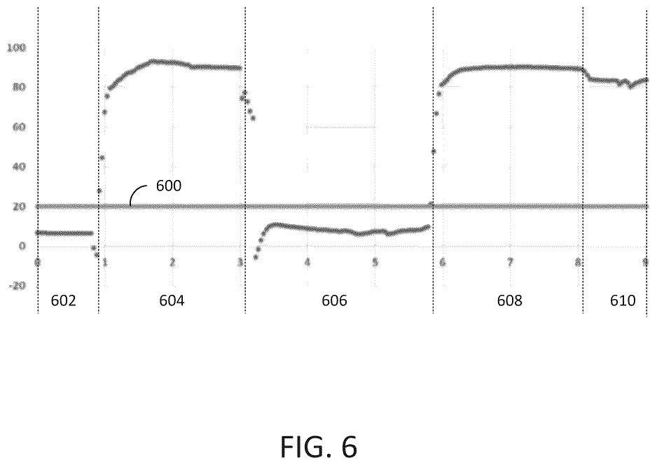

FIG. 6 shows an example plot of a measurement signal of a liquid level sensor at different product levels within a reservoir. In an example embodiment, the measurement signal provides information regarding the level of product in a reservoir relative to the location of first and second sensing areas of a liquid level sensor, such as described herein. In the illustrated embodiment, when the measurement signal is below a predetermined threshold 600, the signal is indicative that the level of the liquid product in the liquid product reservoir is above a first predetermined level (e.g., is sufficiently high so that the liquid product affects the thermal behavior of both the first and second pair of thermistors). Similarly, In the illustrated embodiment, when the measurement signal is above the predetermined threshold 600, the signal is indicative that the level of the liquid product in the liquid product reservoir is below the first predetermined level (e.g., so that the liquid product affects the thermal behavior of one pair of thermistors but not the other pair of thermistors). In the example of FIG. 6, threshold 600 is approximately 20 mV, although other values can be used.

In the illustrated example, in a first time period 602 and a third time period 606, the measurement signal is below the predetermined threshold 600, indicating that the liquid product level is above both pairs of thermistors. During various processes, this can indicate that sufficient product remains in the reservoir for continued use. In some examples, the measurement signal in such a state is approximately 5 mV, though other values are possible.

During a second time period 604 and a fourth time period 608, the measurement signal rises to above the predetermined threshold 600, indicating that the level of product has dropped below a predetermined level. In some examples, dropping below the predetermined level (resulting in the measurement signal to rise above the predetermined threshold 600) indicates that the product level is low and should be refilled or replaced. As described, in some examples, the product level dropping below the predetermined level indicates that sufficient product remains in the reservoir for one more complete process using the product from the reservoir before too little product remains in the reservoir to complete subsequent processes.

During an example system operation, product is extracted from the product reservoir during first time period 602 and the product level falls over time with product use. Once the product falls below a predetermined level (e.g., at the transition from the first time period 602 to the second time period 604), the measurement signal rises above the predetermined threshold 600, for example, due to the product level falling below the second sensing area but not the first sensing area so that the product thermally affects the first pair of thermistors but not the second pair of thermistors. In some embodiments, the measurement signal rises to above 50 mV when the level drops to a level below a first predetermined level and above a second predetermined level (e.g., a level between the first and second sensing areas), though other measurement signal values are possible.

If the reservoir is refilled or replaced such that the product level returns to a level above the predetermined level (e.g., at the transition from the second time period 604 to the third time period 606), the measurement signal falls back below the predetermined threshold 600, indicating sufficient product in the reservoir for operation to continue.

When product in the reservoir is depletable, the level of product may periodically fall below the predetermined level, such as the future transition from the third time period 606 to the fourth time period 608. At this time, the measurement signal again rises above the predetermined threshold 600, which can provide an indication that the reservoir needs to be refilled or replaced.

As described elsewhere herein, in some examples, when the level in the reservoir drops below a second predetermined level, the measurement signal can reach a third value. In some examples, a heat sink (e.g., 340) proximate a first pair of thermistors (e.g., 321) can lead to the thermal behavior similar to that of the case when liquid is present near the first pair of thermistors but not the second. The different thermal behavior in the first pair of thermistors relative to a second pair of thermistors (e.g., 322) creates signals which are above the predetermined threshold 600. Such output signal is shown on the FIG. 6 in the time period 610. In some examples, the effect of the heat sink on the thermal behavior of the thermistors is similar to or less than that of the product in the reservoir when the product is proximate the thermistors. In some such cases, the third value of the measurement signal, corresponding to when the level of product is below both the first and second pairs of thermistors, is between the values of the measurement signals shown in, for example, the first and second time periods. In some examples, the third value of the measurement signal is approximately the same as the in time periods 604 and 608 when the level of product has dropped below a predetermined level, for example, if the heat sink has approximately equal effect on the thermal behavior of the thermistors compared to when fluid is present proximate such thermistors.

In some examples, the measurement signal would reach approximately the value as shown in second time period 604 and fourth time period 608 if the level of liquid product is below the second pair of thermistors, regardless of whether it is below the first pair of thermistors. Even if the measurement signal does not change further after the liquid product level drops below the heat sink and first pair of thermistors, the heat sink can act to ensure that the measurement signal does not revert to below the predetermined threshold 600 if the liquid product level drops below the second sensing area (e.g., due to similar thermal behavior between the first and second pairs of thermistors in the situations when the liquid product level is both above and below both pairs of thermistors). The heat sink prevents such false indications of a fluid level being above both the first and second pairs of thermistors.

Accordingly, the measurement signal in second time period 604 and fourth time period 608 reflect periods of time wherein the liquid product level is at least below a first predetermined threshold corresponding to the second pair of thermistors. In some implementations, such as when the heat sink approximately maintains the thermal behavior of the first pair of thermistors after the liquid level drops below the first sensing area, the measurement signal in second time period 604 and fourth time period 608 can reflect periods of time when the liquid product level is either above or below the first sensing area.

While the system shown in FIG. 1B shows a plurality of liquid level sensors (123A . . . 123N) on a corresponding plurality of reservoirs (103A . . . 103N), in some examples multiple liquid level sensors can be used in a single reservoir. For instance, in some examples, a first liquid level sensor can be configured to detect when a level of liquid product drops below a first level (e.g., below the second sensing area of the first sensor) and a second liquid level sensor can be configured to detect when the level of liquid product drops below a second level (e.g., below the second sensing area of the second sensor). In some examples, dropping below the first level corresponds to a warning, such as a reservoir containing enough product to run a predefined process a certain number of times (e.g., five times), and dropping below the second level corresponds to a more urgent warning, such as the reservoir containing enough product to run the predefined process one more time.

In various implementations, liquid level sensors can be positioned provide information about the level of liquid product in a reservoir dropping below any number of levels. Predetermined levels can be pre-set (e.g., by choosing a relative position within a reservoir to position one or more liquid level sensors) according to a desired installation, such as designating a number of uses worth of a liquid product remaining in a reservoir before the liquid level sensor outputs an indication of the level of liquid product reaching such a level.

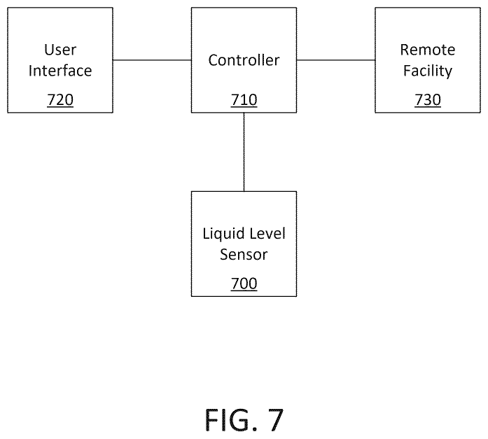

A system including a liquid level sensor can output an alert based on a liquid level in a variety of ways. FIG. 7 shows an example system including a liquid level sensor and configured to provide an indication of a level of liquid in a product reservoir. The example of FIG. 7 includes a liquid level sensor 700 in communication with a controller 710. In some cases, such as described elsewhere herein, the controller 710 can receive a measurement signal from the liquid level sensor 700 (e.g., the measurement signal shown in FIG. 6) that provides information regarding a level of liquid product in a reservoir.

The controller 710 is in communication with a user interface 720 and a remote facility 730 (e.g., via a network). The controller 710 can be configured to output an indication based on the measurement signal from the liquid level sensor 700. For instance, in some examples, controller 710 can be configured to determine, based on the measurement signal, if the level of liquid product in the reservoir drops below a predetermined level, and, if so, can output an indication. Similar to as described herein, in some examples, the level of liquid product dropping below a predetermined level corresponds to a measurement signal rising above a predetermined threshold (e.g., during second time period 604 in FIG. 6). The controller 710 can be configured to output an indication via the user interface 720 indicating that the liquid level is below a predetermined level if the measurement signal rises above a predetermined threshold. The user interface 720 can include a visual interface, such as a display or one or more lights that can be illuminated, and/or an audio interface, such as a speaker configured to output an audible alert. As described, in some cases, the indication output via a user interface 720 can inform a local user that the level of product has dropped to or below a predetermined level, and the reservoir should be refilled or replaced.

Additionally or alternatively, the controller 710 can output an indication to the remote facility 730 indicating that the liquid product level has dropped to or below a predetermined level. The remote facility 730 can include a central monitoring facility that can initiate a refill or replacement of the product reservoir. For example, in some implementations, a supplier at a remote facility 730 can proactively monitor a level of liquid product in a reservoir and replace the reservoir with a new one, which can help to prevent product shortages and ensures continuous and efficient use of the product.

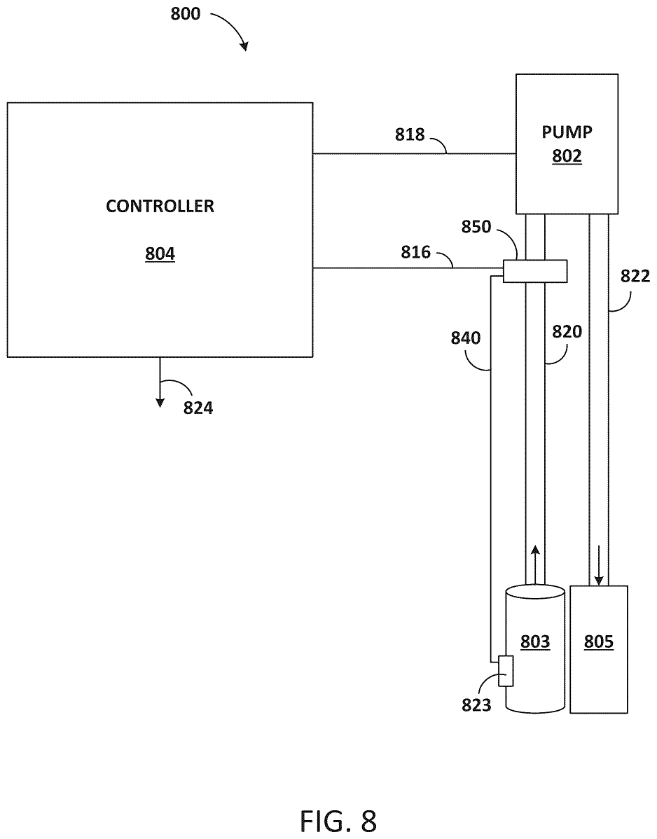

Liquid level sensors such as those described herein can be used together with different OOP sensors. There are some OOP sensors such as a heat transfer OOP sensors that can provide not only information about present or absence of a product in a product line but also information about product flow, having distinctly different output signals indicating separate FLOW and NO FLOW (e.g., product is present in but not flowing through the product line) states. The use of a liquid level sensor with such OOP sensor provides additional advantages allowing verification of product delivery. Sample of such product delivery system 800 is shown in FIG. 8.

FIG. 8 is a diagram illustrating an example fluid flow system with a liquid level sensing system that detects a relative level of a product to be dispensed and an out of product (OOP) sensor configured to detect a presence of a flowing liquid product.

A liquid level sensor 823 based on heat transfer method can be used in conjunction with an OOP sensor 850 also based on heat transfer method. In an example embodiment, the liquid level sensor 823 produces the signal HIGH when the level in the reservoir 803 is above a selected detection level and produces the signal LOW when the liquid level in the container is below the selected detection level. The heat transfer OOP sensor 850 allows detection of three distinct states in the line 820. Accordingly, three different signals output from the OOP sensor 850 can correspond to different possible flow status in the line 820: NO PRODUCT, NO FLOW (product present, but not flowing in line), FLOW. The liquid level sensor 823 can be connected electrically to OOP sensor by line 840. In some examples, the OOP sensor 850 comprises a thermistor bridge similar to that shown in FIG. 4 and can be used by applying excitation pulses and measurement pulses such as shown and described with respect to FIGS. 4 and 5. Line 840 connecting liquid level sensor 823 and OOP sensor 850 allows liquid level sensor 823 and OOP sensor 850 to share some pulse excitation electronics and ADC readings channels, allowing for a lower cost installation for using both sensors.

In the example of FIG. 8, the signals HIGH, LOW, NO PRODUCT, NO FLOW, FLOW can be delivered by connection line 816 to a controller 804, wherein the controller 804 has connection line 818 to a product pump 802. The pump 802 delivers product from the reservoir 803 to target 805 (which can be dish machine, laundry machine, cleaning equipment, sprayers, mixer for dilution of use solution, etcetera). The controller 804 can be configured to analyze signals from the liquid level sensor 823 and OOP sensor 850 and control the operation of the product pump 802. Signals from controller can be sent to next level system using output line 824.

The use of heat transfer sensors as on FIG. 8 can more accurately offer a proof of delivery than just one sensor in the system. If the output of the liquid level sensor is HIGH and the OOP sensor is FLOW, then the system can confirm with a high certainty that the product was delivered to target 805. In some embodiments the OOP sensor can be moved to the outlet line that is located between the pump 802 and the target 805.

The product delivery monitoring system 800 shown on the FIG. 8 provides a way to evaluate several scenarios allowing to understand the delivery system errors and to help with analysis. The controller 804 can receive signals from sensors 823 and 850 and direct a user to specific troubleshooting steps for those detected scenarios based on the signals from level sensor 823 and OOP sensor 850.

In one example, the delivery system 800 is actively trying to power the pump and the liquid level sensor 823 reports HIGH (e.g., above first and second sensing areas) but OOP sensor 850 reports NO FLOW then the system could determine that there is an issue with the pump, control system, or wiring.

In another example, the delivery system 800 is actively trying to power the pump and the liquid level sensor 823 reports HIGH but OOP sensor 850 reports NO FLOW for limited time and then reports FLOW, the system could determine that there is a priming issue, and the product dosing time should be counted from the moment the OOP sensor 850 start showing FLOW. The ability to detect the priming error is very useful for applications where products demonstrate off gassing (e.g., some products in dishwashing applications). When a product demonstrates off gassing, a pump trying to pump the product can be empty for some length of tubing as the pump moves the gas rather than a liquid product, creating a delay before product reaches the pump and the delivery system starts dosing of a product. Confirming delivery of product only begins when the OOP sensor starts showing a FLOW status can be important to maintain a required concentration of chemicals in point of delivery at target 105.

In another example, the delivery system 800 is actively pumping a product wherein the liquid level sensor 823 reports HIGH and OOP sensor 850 reports FLOW indicating normal product delivery. If at some moment the liquid level sensor 823 starts reporting LOW and OOP sensor 850 reports FLOW, the controller can output a first warning signal from the system indicating that the product level is low and only limited amount of the product available. A warning about future shortage can be sent allowing time to deliver new batch of product. The liquid level sensor 823 positioning in the product reservoir 803 could be adjustable to provide the desired level for the LOW Product state depending on the typical dosage sizes of the given customer and installation. If after first signal the liquid level sensor 823 reports LOW and OOP sensor 850 start reporting NO PRODUCT or NO FLOW, the system can output a second warning signal indicating that there is no product at the pump input and the operation of equipment should be stopped.

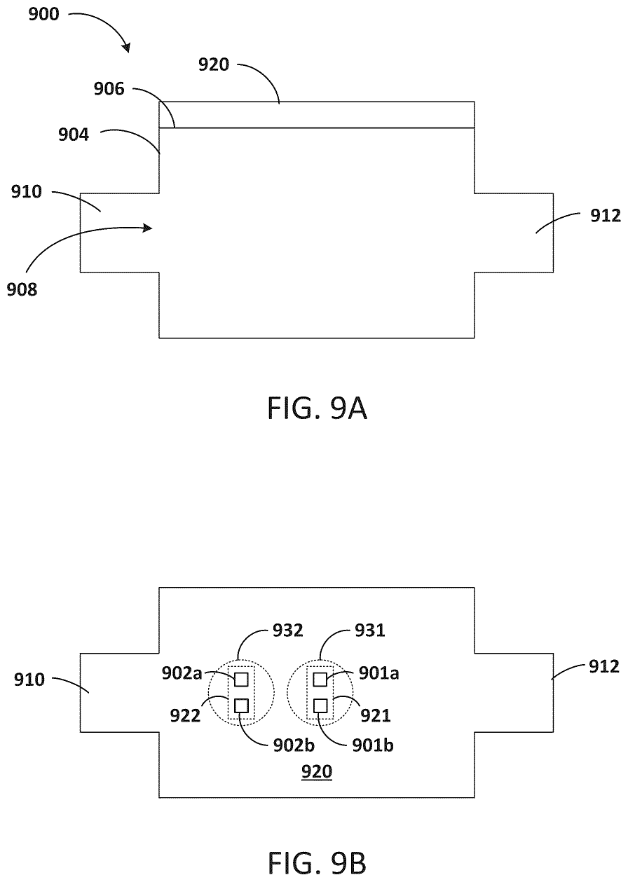

In some embodiments, an OOP sensor uses heat transfer to a fluid in a fluid line and an OOP thermistor bridge for detecting flow states of fluid through the OOP sensor. FIG. 9A shows a side view of an example configuration of an OOP sensor. In the illustrated example, OOP sensor 900 comprises a housing 904 having a first surface 906 and defining a flow channel 908 through which a fluid can flow. The sensor 900 comprises an inlet 910 and an outlet 912, each fluidly connecting the flow channel 908 to an exterior of the housing 904. In some examples, the inlet 910 and outlet 912 are configured to couple to tubing that can carry fluid to and from the sensor 900 such that the fluid flows from a tubing, through the inlet 910, through the flow channel 908, through the outlet 912, and into additional tubing. With reference to FIG. 8, in some examples, tubing can connect inlet 910 and a reservoir (e.g., 803), and additional tubing can connect outlet 912 to a dispensing site (e.g., 805). In some examples, a pump (e.g., 802) can be positioned between such a reservoir (e.g., 803) and inlet 910 and/or between outlet 912 and such a dispensing site (e.g., 805).

The sensor 900 includes a circuit board 920 coupled to the first surface 906 of the housing 904. In some examples, the circuit board 920 supports an OOP thermistor bridge, which can be arranged similar to the thermistor bridge 10 shown in FIG. 4. In some embodiments, fluid flowing through the flow channel 908 (e.g., a liquid product from a product reservoir) thermally interacts with portions of the OOP thermistor bridge supported by the circuit board 920 such that the fluid affects the temperature of one or more OOP thermistors of the OOP thermistor bridge. In some examples, an air gap is provided around OOP thermistors to prevent heat loss from the OOP thermistors to other structures of an OOP sensor.

FIG. 9B shows a top view of the OOP sensor of FIG. 9A. In the example of FIG. 9B, OOP sensor 900 includes an inlet 910 and an outlet 912, such as described with respect to FIG. 9A. The OOP sensor comprises a circuit board 920 supported by a first surface of the housing of the OOP sensor 900. In the example, the circuit board 920 supports first 901a, second 902b, third 902a, and fourth 901b OOP thermistors. As noted in the example of FIG. 4, in some embodiments, the first OOP thermistor 901a and the fourth OOP thermistor 901b form a first pair of OOP thermistors 921 and the second OOP thermistor 902b and the third OOP thermistor 902a form a second pair of OOP thermistors 922. In some embodiments, the first pair of OOP thermistors 921 are positioned in a first OOP sensing area 931 and the second pair of OOP thermistors 922 are positioned in a second OOP sensing area 932. In some such embodiments, the housing 904 is configured such that the thermal resistance between the flow channel of the OOP sensor 900 and the first OOP sensing area 931 is lower than the thermal resistance between the flow channel of the OOP sensor 900 and the second OOP sensing area 932. In some such examples, fluid (e.g., a liquid from a product reservoir) flowing in the flow channel of the OOP sensor will have a greater impact on the temperature of the first pair of OOP thermistors 921 compared to the second pair of OOP thermistors 922. And in some examples, fluid flowing in the flow channel 908 (e.g., a liquid product) will affect the thermal behavior of the first pair of OOP thermistors compared to an absence of the fluid (e.g., when only air is present in the flow channel). And in some examples, fluid present but not flowing in the flow channel 908 will affect the thermal behavior of the first pair of OOP thermistors 221 more than the absence of fluid (e.g., when only air is preset) but less than a flowing fluid. In some examples, the presence of fluid, whether flowing or not, does not significantly affect the thermal behavior of the second pair of OOP thermistors 922.

Various configurations are possible to cause the fluid in the flow channel to affect the thermal behavior of the first pair of OOP thermistors 921 compared to the second pair 922. For instance, circuit board 920 can be curved such that the first pair of OOP thermistors 921 are closer to the flow channel 908 than the second pair of OOP thermistors 922. Additionally or alternatively, the flow channel can include an aperture that allows fluid in the flow channel 908 to contact the circuit board proximate the first pair of OOP thermistors 921 and not the second pair of OOP thermistors 922. Additionally or alternatively, the flow channel 908 can be curved toward the circuit board 920 near the first OOP sensing area 931 to direct fluid in the flow channel 908 to more effectively thermally affect the thermal behavior of the first pair of OOP thermistors 921 compared to the second pair of OOP thermistors 922. Various configurations are possible and are described in U.S. patent application Ser. No. 18/830,421, filed Sep. 10, 2024, which is assigned to the assignee of the instant application and which is incorporated herein by reference.

In some examples, when a fluid in the flow channel 908 affects the first pair of OOP thermistors 921 more than the second pair of OOP thermistors 922, differences in temperature of various OOP thermistors can cause changes in voltages across the OOP thermistor bridge when a measurement pulse is applied. Higher magnitude volage differences across the OOP thermistor bridge can indicate larger temperature differences between the first pair of OOP thermistors 921 more than the second pair of OOP thermistors 922.

While described as showing top and side views in FIGS. 9A and 9B, respectively, such labels are used for ease of reference and do not limit the orientation in which the OOP sensor 900 can be used during operation. In some examples, inlet 910 and outlet 912 are aligned vertically such that fluid flows upward through flow channel 908. Other orientations are possible.

In some examples, excitement and measurement pulses can be provided to the OOP thermistor bridge of the OOP sensor 900 similar to those shown in FIG. 5 to produce a measurement signal representative of a voltage between sides of the OOP thermistor bridge (e.g., between points like 11 and 12 of thermistor bridge 10 in FIG. 4). The value and/or pattern of the measurement signal can be used (e.g., by controller 804) to determine a flow state of fluid through the OOP sensor. Various detectable states can include FLOW, NO FLOW, and NO PRODUCT such as discussed above with respect to FIG. 8.

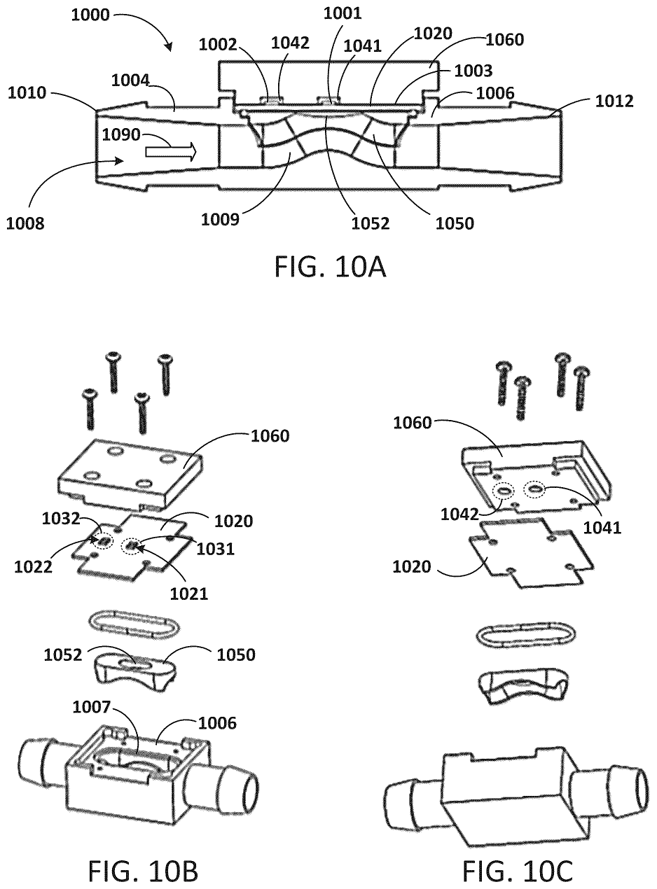

FIG. 10A shows an example cross-sectional view of an embodiment of an OOP sensor. The OOP sensor 1000 of FIG. 10A includes a housing 1004, a flow channel 1008, an inlet 1010 and an outlet 1012 that each coupled the flow channel 1008 to an exterior of the housing 1004. In some examples, OOP sensor 1000 is configured such that fluid flows in the direction of arrow 1090. During example operation, OOP sensor can be oriented such that arrow 1090 points upward and fluid flows vertically upwards through flow channel 1008, though operating in other orientations is possible. The housing includes a first surface 1006, and the OOP sensor 1000 includes an OOP circuit board 1020 supported by the first surface of the housing.

In the example of FIG. 10A, flow channel 1008 comprises a bend 1009 toward the OOP circuit board 1020. The bend 1009 can be configured to cause fluid flowing through the flow channel 1008 to be directed more toward certain portions of the OOP circuit board 1020 than others. In some embodiments, the bend 1009 in flow channel 1008 directs fluid more toward a first OOP sensing area, where a first pair of OOP thermistors are located, compared a second OOP sensing area, where a second pare of OOP thermistors are located.

FIG. 10B shows a perspective exploded view of the OOP sensor of FIG. 10A. As shown, OOP circuit board 1020 is configured to engage first surface 1006 of housing 1004. In some examples, OOP circuit board is between approximately 0.025 mm thick and 0.2 mm thick. In some examples, the OOP circuit board comprises a glass epoxy laminate FR-4 or polyimide.

OOP sensor 1000 includes an insert 1050, for example, a plastic insert, configured to be inserted into an aperture 1007 in the first surface 1006 of the housing 1004. In some examples, the insert engages with a portion of the flow channel 1008. For instance, in some embodiments, the housing 1004 defines a first half of a tubular flow channel 1008 and the insert 1050 comprises an inner surface defining a second half of a tubular flow channel. In some such embodiments, when the insert is inserted into the aperture in the first surface 1006 of the housing 1004, the flow channel defined by the housing 1004 and the inner surface of the insert 1050 join to form a closed tubular flow channel.

The insert 1050 as shown includes an aperture 1052 extending therethrough. In some examples, the aperture 1052 provides a fluid path between the flow channel 1008 and the OOP circuit board 1020 when the OOP sensor 1000 is assembled.

OOP circuit board 1020 includes a first pair of OOP thermistors 1021 (e.g., including OOP thermistor 1001) located in a first OOP sensing area 1031 and a second pair of OOP thermistors 1022 (e.g., including OOP thermistor 1002) located in a second OOP sensing area 1032. In an example embodiment, when OOP circuit board 1020 engages the first surface 1006 of the housing 1004 (e.g., attached via threaded bolts configured to extend through corresponding holes in the OOP circuit board 1020 and engage corresponding threaded holes in the housing 1004), the first OOP sensing area 1031 is positioned over the aperture 1052 in the insert 1050 that forms a second half of the flow channel 1008 at bend 1009, while second OOP sensing area 1032 is positioned closer to the inlet 1010 and is not positioned over the aperture 1052. In some examples, bend 1009 directs fluid flowing in flow channel 1008 toward the first OOP sensing area 1031, but does not direct fluid toward the second OOP sensing area 1032. Additionally or alternatively, the aperture 1052 in the insert 1050 allows fluid in the flow channel 1008 to reach the OOP circuit board 1020 at the first OOP sensing area 1031 while preventing the fluid in the flow channel 1008 from reaching the OOP circuit board 1020 at the second OOP sensing area 1032. Thus, in some examples, fluid flowing through the flow channel 1008 will have a larger effect on the temperature of OOP thermistors in the first OOP sensing area 1031 compared to the second OOP sensing area 1032.

For example, FIG. 10A shows an OOP thermistor 1001 in a first OOP sensing area over aperture 1052 in insert 1050 and an OOP thermistor 1002 in a second OOP sensing area not over aperture 1052. In some embodiments, the insert 1050 provides thermal insulation between the flow channel 1008 and OOP thermistor 1002, but not between flow channel 1008 and OOP thermistor 1001 due to aperture 1052.

In some examples, a protective layer is positioned between OOP thermistors in the first OOP sensing area 1031 (e.g., in the first pair of OOP thermistors 1021 in the first OOP sensing area 1031) and flow channel 1008. In some examples, the OOP thermistors are positioned on a first surface of the OOP circuit board 1020 such that the OOP circuit board 1020 is between the OOP thermistors and the flow channel 1008. In some such examples, the OOP circuit board 1020 serves as the protective layer. In other examples, the OOP thermistors are positioned on an inner surface of the OOP circuit board 1020 such that the OOP thermistors face the flow channel 1008. In some examples, an acrylic or Teflon protective layer can be positioned over the OOP thermistors to form a protective layer between the OOP thermistors and the flow channel 1008.

FIG. 10C shows another perspective exploded view of the OOP sensor of FIG. 10A.

In some examples, OOP sensor 1000 includes a cap 1060 configured to be coupled to the housing 1004, for example, by one or more bolts (e.g., via threaded bolts configured to extend through corresponding holes in the OOP circuit board 1020 and the cap 1060 and engage corresponding threaded holes in the housing 1004). In some examples, cap 1060 is placed over the OOP circuit board 1020 such that an inner surface of the cap 1060 faces the OOP circuit board 1020. The inner surface of the cap 1060 can include cavities to accommodate OOP thermistors on the OOP circuit board. In the example of FIG. 10, cavities 1041 and 1042 can be positioned such that, when the cap 1060 is placed over the OOP circuit board 1020, the first pair of OOP thermistors 1021 are received in cavity 1041 and the second pair of OOP thermistors 1022 are received in cavity 1042. Cavities can provide air gaps around the OOP thermistors to prevent heat loss from the OOP thermistors, for example, by conduction of heat to cap 1060 or other components of the OOP sensor 1000.

In some embodiments, a controller (e.g., 804) can be programmed with one or more thresholds including, for example, first and second predetermined thresholds corresponding to detecting the presence or absence of fluid. Additionally or alternatively, in some embodiments, a measurement signal from the OOP sensor can be analyzed to provide additional information about the system. For instance, a measurement signal can provide information representative of poor pump operation, broken tubing, bubbles present in the line, or other flow system characteristics. In some embodiments, a controller can be configured to analyze the measurement signal in order to detect one or more such occurrences.

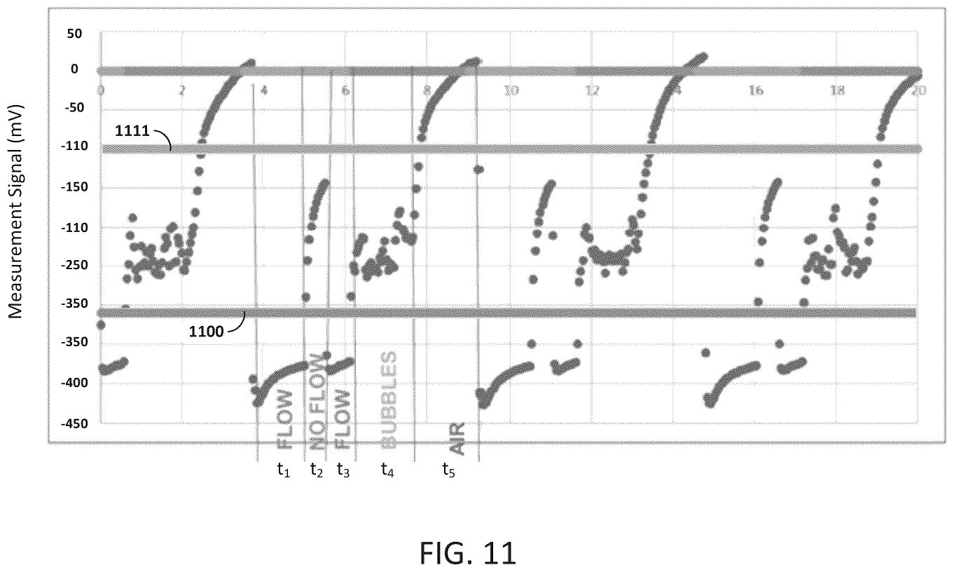

FIG. 11 shows an example plot of a measurement signal over time that can be used to detect the presence of flowing fluid, fluid that is not flowing, no fluid (e.g., air only) and fluid with bubbles in the OOP sensor. As shown in the illustrated example, in some embodiments, when a measurement signal is below a first predetermined threshold value 1100, the controller can determine that fluid is flowing in the flow channel, such as during time periods t₁ and t₃ of FIG. 11. When a measurement signal is above a second predetermined threshold value 1110, the controller can determine that no fluid is present in the flow channel, such as in time period t₅. When the measurement signal is between the first predetermined threshold value 1100 and the second predetermined threshold value 1110, the measurement signal can be analyzed to determine further information about the flow status. For instance, the measurement signal pattern in time period t₂ can be representative of fluid present, but not flowing in the flow channel, while the measurement signal pattern in time period t₄ can be indicative of a fluid with bubbles flowing through flow channel. The controller can be configured to recognize patterns in the measurement signal and output information regarding the flow status, such as the presence of bubbles in the flow channel. In some examples, a controller does not detect bubbles, but instead interprets a measurement signal between the first 1100 and second 1110 predetermined thresholds as indicating a present but not flowing liquid within the flow channel.

Various OOP measurement signal thresholds such as shown and described with respect to FIG. 11 can be used in conjunction with determinations regarding the liquid level sensor to gain additional information about the system status.

Various illustrative examples have been described. These and others are within the scope of this disclosure. The following list of enumerated embodiments illustrate some aspects of the disclosure.

1. A liquid level sensing system comprising:
a liquid level sensor comprising:
a circuit board;
a thermistor bridge supported by the circuit board, the thermistor bridge comprising a first pair of thermistors and a second pair of thermistors; and
a heat sink coupled to the circuit board proximate the first pair of thermistors and not the second pair of thermistors;
a power supply configured to provide pulses of electrical current through the thermistor bridge; and
a controller in communication with the thermistor bridge, wherein the controller is configured to:
receive a measurement signal from the thermistor bridge; and
output a low product indication when the measurement signal satisfies a predetermined condition.

2. The system of claim 1, wherein the pulses of electrical current comprises pulse durations from 0.1 ms to 10 ms.

3. The system of claim 2, wherein the pulses of electrical current generate a self-heating of thermistors in the thermistor bridge and increase the temperature of thermistors during the pulses of electrical current by a preselected value.

4. The system of claim 1, further comprising a current limiting resistor connected between the power supply and the thermistor bridge.

5. The system of claim 1, wherein the predetermined condition comprises the measurement signal being above a predetermined threshold so that the controller is configured to output the low product indication when the measurement signal is above the predetermined threshold.

6. The system of claim 5, wherein the controller is configured to output a second indication and not the low product indication when the measurement signal is below the predetermined threshold.

7. The system of claim 5, wherein:
the predetermined threshold comprises a first predetermined threshold, and wherein the controller is further configured to:
output a second indication different from the low product indication when the measurement signal is below a second predetermined threshold, the second predetermined threshold being lower than the first predetermined threshold; and
output a third indication different from the second indication when the measurement signal is between the first predetermined threshold and the second predetermined threshold.

8. The system of claim 1, wherein:
the thermistor bridge comprises a first branch having a first thermistor in series with a second thermistor and a first point between the first thermistor and the second thermistor and a second branch having a third thermistor in series with a fourth thermistor and a second point between the third thermistor and the fourth thermistor, the first branch and the second branch being arranged in parallel between a powered side of the thermistor bridge and a reference side of the thermistor bridge such that the first thermistor and the third thermistor are coupled to the powered side of the thermistor bridge and the second thermistor and the fourth thermistor are coupled to the reference side of the thermistor bridge;
the first pair of thermistors comprises the first thermistor and the fourth thermistor;
the second pair of thermistors comprises the second thermistor and the third thermistor; and
the measurement signal is based on a voltage between the first point and the second point.

9. The system of claim 8, further comprising a power supply in communication with the thermistor bridge; and wherein
the controller is in communication with the power supply and is further configured to cause the power supply to provide electrical current to the powered side of the thermistor bridge.

10. The system of claim 1, wherein outputting the low product indication comprises sending an alert to a remote monitoring facility.

11. The system of claim 1, wherein the heat sink is positioned on an opposite side of the circuit board compared to the first pair of thermistors.

12. The system of claim 1, wherein the liquid level sensor comprises a housing defining an interior, wherein the circuit board is coupled to the housing such that the first pair of thermistors and the second pair of thermistors are within the interior of the housing.

13. A liquid level sensing system comprising:
a reservoir configured to hold a liquid product; and
a liquid level sensor comprising a circuit board and a thermistor bridge supported by the circuit board, the thermistor bridge comprising a first pair of thermistors located in a first sensing area and a second pair of thermistors located in a second sensing area different from the first sensing area;
wherein the liquid level sensor is configured relative to the reservoir such that:
if a level of liquid product in the reservoir is above a first predetermined level, the liquid engages the first sensing area and the second sensing area;
if the level of liquid product in the reservoir is below the first predetermined level but above a second predetermined level, the second predetermined level corresponding to less product in the reservoir compared to the first predetermined level, the liquid product engages the first sensing area but not the second sensing area; and
if the level of liquid product in the reservoir is below the second predetermined level, the liquid product does not engage the first sensing area or the second sensing area.

14. The system of claim 13, further comprising a controller configured to receive a signal from the liquid level sensor indicative of the level of liquid product in the reservoir.

15. The system of claim 14, further comprising:
a pump;
an input tubing connecting the reservoir to the pump;
output tubing connected to the pump such that the pump causes liquid product to flow from the reservoir, through the input tubing to the pump and from the pump to the output tubing; and
an out of the product (OOP) sensor installed in the input tubing and configured to determine a flow state of liquid product flowing through the input tubing.

16. The system of claim 15, wherein the OOP sensor comprises a flow channel coupled to the input tubing and an OOP thermistor bridge, and wherein the OOP sensor is configured to output an OOP measurement signal based on a heat transfer between the OOP thermistor bridge, the OOP sensor being configured to output different OOP measurement signal values to the controller indicating (i) no product in the flow channel, (ii) product flowing in the flow channel, or (iii) product present but not flowing in the flow channel.

17. The system of claim 16, wherein the controller is configured to detect a fluid delivery failure based on the signal from the liquid level sensor indicative of the level of liquid product in the reservoir and the OOP measurement signal.

18. The system of claim 14, wherein the controller is configured to determine, based on the signal received from the liquid level sensor, the level of liquid product in the reservoir relative to the first predetermined level.

19. The system of claim 13, wherein the liquid level sensor is positioned on a wall of the reservoir.

20. The system of claim 13, wherein the circuit board has a first side and a second side, opposite the first, and wherein the first pair of thermistors and the second pair of thermistors are positioned on a first side of the circuit board and the liquid level sensor is positioned such that the second side of the circuit board is exposed to the liquid product in the reservoir.
